(19) United States
(12) Patent Application Publication
(10) Pub. No.: US 2005/0096670 A1
(43) Pub. Date: May 5, 2005

(54) Surgical end effector

(76) Inventors: Parris Wellman, Hillsborough, NJ (US); Simon Cohn, Rutherford, NJ (US); John Young, Staten Island, NY (US); Joshua Samon, Jersey City, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/699,280

(22) Filed: Oct. 31, 2003

(51) Int. Cl. A61B 17/10

(52) U.S. Cl. 606/139

(57) ABSTRACT

A surgical end effector includes an anvil movable between a first position and a second position, the anvil having a concave anvil surface, and a convex mating surface. The anvil clamps a tissue structure between the anvil surface and the mating surface when in the second position, and wherein the mating surface has a different radius of curvature than the anvil surface.

### FIELD OF THE INVENTION

The present invention relates generally to surgical devices, and more particularly, to a surgical device for clamping, ligating, and severing tissue, preferably, a side branch of a vessel to be harvested.

### BACKGROUND OF THE INVENTION

Endoscopic vessel harvesting (EVH), particularly of the greater saphenous vein in the leg and the radial artery in the arm, is a surgical procedure for obtaining a graft vessel for a coronary artery bypass graft (CABG) procedure. A physician's assistant (PA) typically performs the EVH on one or both legs and/or arms of the patient by operating endoscopically with instruments actuated at a position remote from the operating site to harvest saphenous veins and/or radial arteries.

Conventional techniques for harvesting these vessels involve an incision length approximately equal to the length of the vessel being harvested. More recently, various bipolar endoscopic vessel-harvesting devices have been developed for removing saphenous veins or radial arteries in a minimally invasive manner. See, e.g., U.S. Pat. No. 6,464,702 (Schulze), U.S. Pat. No. 6,206,823 (Kolata), U.S. Pat. No. 5,902,315 (Dubois), and U.S. Patent Application Publication No. 2003/0065348 (Hess), each of which is hereby incorporated by reference. Known methods and devices for performing vessel dissection are discussed in detail in U.S. Pat. No. 5,667,480 (Knight) and U.S. Pat. No. 5,722,934 (Knight), both of which are incorporated herein by reference.

One example of such a device is disclosed in U.S. Pat. No. 5,928,138 (“Method and Devices for Endoscopic Vessel Harvesting”, assigned to Ethicon Endo-Surgery, Inc., and issued on Jul. 27, 1999) discloses an optical retractor/dissector having a concave working head. A commercial version of this optical dissector is called the CLEARGLIDE® system and is available from Ethicon, Inc., Somerville, N.J. The CLEARGLIDE system provides good access and visibility to the surgical site along the greater saphenous vein. When using the CLEARGLIDE system, the PA typically also uses other endoscopic, surgical dissection instruments to isolate the vessel from surrounding tissues. The PA introduces these instruments beneath the shaft of the CLEARGLIDE retractor so as to position the end effector of the instrument within a working space created by the retractor to operate on tissues.

Still yet another approach involves the use of scissor-like clamping jaws that open around a side branch, and then must be closed, at which time an electrical current is applied to the vessel within the jaws before the vessel is harvested. These types of instruments, however, are difficult to use in confined spaces because the upward opening movement of at least one of the jaws often interferes with objects in the field. Further, the upward opening jaw obscures the user's field of vision.

Users of current devices frequently struggle to separate side branches of the veins or arteries when a side branch run beneath (posteriorly) or above (anteriorly) the main trunk of the vessel. In addition, current devices and methods for endoscopic vessel harvesting that use mechanical tissue retraction require the user to have great dexterity. Normally, one hand manipulates the tissue retractor, while another hand manipulates one or more tools to perform side branch hemostasis, transection and verification of side branch transection. This set of tools provides the user with great flexibility when the procedure requires the user to access difficult-to-reach areas. The skills required to manipulate multiple tools simultaneously, however, take some time to refine, and are difficult to master for novice users and those who do not have innate, hand-eye coordination.

In addition to vessel harvesting procedures, many other surgical procedures require cutting of tissue and control of the bleeding from the cut tissue. In fact, many surgical instruments are commercially available that cut and desiccate tissue (i.e., bipolar scissors, harmonic scissors). These instruments, however, are not well suited for desiccation without clamping or cutting the tissue; i.e. they do not provide the ability to spot coagulate.

In the design of surgical tools, it is often desirable to produce large amounts of force with small button actuation forces. Tools that provide such a feature typically achieve it with mechanisms using mechanical advantage. Unfortunately displacement is traded for force in such mechanisms, and given the limited space typically available for mechanisms of this type in hand tools, such a tradeoff can pose a problem. For example, in the case of bipolar surgical forceps or other clamping instruments, it is often desirable to be able to provide a large amount of force to close the jaw, and yet also be able to provide a large displacement to open the jaw. That is, it is desirable to have a mechanism that provides high force amplification in one direction and 1:1 displacement in the other. Levers, gears and cam mechanisms have also been used for this purpose. The problem with these fixed ratio mechanisms is that the employ the same motion ratio in both directions. For instance, if a mechanism is designed that provides a ten-fold increase in force, it requires a ten-fold increase in displacement. Thus, to provide a jaw that opens twenty millimeters would require 200 millimeters of button travel, a length typically not available on most hand tools.

### SUMMARY OF THE INVENTION

Therefore it is an object of the present invention to provide instruments and methods for their use that overcome the disadvantages of conventional instrumentation known in the art.

The system according to the present invention is a set of two instruments. A retractor is used primarily for gross tissue retraction, but also provides for fine tissue manipulation using thumb-activated controls. A multitool instrument provides a means for endoscopic visualization, side branch hemostasis, and transection. The tools can be used independently or together. A docking feature located on the multitool allows the retractor and the multitool instrument to be docked together, thereby making the two instruments act as one.

Accordingly, a surgical device for severing tissue is provided. The surgical device includes a shaft having a lumen and an opening disposed at a distal end, the shaft movable between a rear position and a forward position, an anvil slidingly disposed in the opening between open and closed positions to capture tissue within the opening, at least one electrode for applying RF energy to the tissue captured in the opening, and an actuator operatively connected to the shaft for moving the shaft between the rear position and the forward position.

Also provided is a surgical system that includes a shaft having a lumen and an opening disposed at a distal end, a tip disposed at the distal end of the shaft, the tip having a slot, a cutting blade slidingly disposed in the opening between an open position and a closed position, the cutting blade having a cutting edge to sever the tissue disposed in the opening, the cutting blade further slidable from the closed position to a forward position whereat the cutting edge is distal to the tip, and an actuator operatively connected to the cutting blade for moving the cutting blade between the open position and the closed position and between the closed position and the forward position.

Also provided is a method for severing tissue with the surgical devices of the present invention. The method includes the steps of: providing a surgical device having a shaft having a lumen and an opening disposed at a distal end, a tip disposed at the distal end of the shaft, the tip having a slot, a cutting blade slidingly disposed in the opening between an open position and a closed position, the cutting blade having a cutting edge to sever the tissue disposed in the opening, the cutting blade further slidable from the closed position to a forward position whereat the cutting edge is distal to the tip, the cutting blade being electrically connected to a source of RF energy, and an actuator operatively connected to the cutting blade for moving the cutting blade between the open position and the closed position and between the closed position and the forward position; capturing tissue in the opening; sliding the cutting blade from the open position to the forward position such that at least a cutting edge is disposed distal to the tip; and applying RF energy with the cutting edge of the cutting blade to cauterize tissue located distal to the tip.

This invention will permit, with one tool, the user to clamp, desiccate, and cut tissue, while also permitting the user to cut and desiccate tissue without clamping within the jaws (i.e. spot coagulation).

Also provided is a mechanism that provides high force amplification in one direction and direct displacement coupling in the other. The mechanism has directional stiffness and direction force multiplication. In one direction, the mechanism provides high force amplification, and in the other direction low amplification with direct coupling of motion. The forces applied, and the impedance are individually adjustable, and can be set for a particular mechanism. This is particularly useful in the clamping, cutting and coagulating instrument being developed for endoscopic vessel harvesting, but is not limited to such an instrument.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 is a perspective view of the endoscopic system including a retractor and multitool device in an undocked configuration;

FIG. 1A is a rear view of the retractor of FIG. 1;

FIG. 2 is a perspective view of the endoscopic system including the retractor and multitool device in a docked configuration;

FIG. 3 is a perspective view of a preferred implementation of a retractor of the present invention;

FIG. 4 is a perspective view of the retractor of FIG. 3, the retractor having a first paddle in an extended position;

FIG. 5 is a perspective view of the retractor of FIG. 3, the retractor having a first and second paddle in an extended position;

FIG. 5A is sectional view of the retractor shown in FIG. 5 taken along line 5A-5A;

FIG. 6 is a sectional view of the retractor shown in FIG. 3 taken along line 6-6;

FIG. 7 is a sectional view of the retractor shown in FIG. 4 taken along line 7-7;

FIG. 8 is a sectional view of the retractor shown in FIG. 5 taken along line 8-8;

FIG. 9 is a side view of the retractor shown in FIG. 4;

FIG. 10 is a side sectional view of the retractor shown in FIG. 3;

FIG. 11 is an exploded view of the retractor shown in FIG. 3 with the handle omitted for clarity;

FIG. 12 is an exploded view of the retractor handle shown in FIG. 3;

FIG. 13 is an exploded view of the multitool device shown in FIG. 1;

FIG. 14 is a perspective view of the handle and actuation system of the multitool device of FIG. 1 with the top half of the handle rotated off of the bottom half of the handle;

FIG. 15 is a perspective view of one embodiment of the dock and dock port of the invention in a docked configuration;

FIG. 16 is a side view of the retractor and multitool device shown in FIG. 2 in a docked configuration;

FIG. 17 is a perspective view of the distal end of the surgical device and end tip;

FIG. 18 is a perspective view of the tip of the surgical device;

FIG. 19 is an exploded view of the anvil assembly of the surgical device;

FIGS. 20a-d are graphical representations of an anvil acting on a surface and the resulting stress diagrams for three different anvil configurations;

FIG. 21 is an exploded view of the sled of the multitool actuation system;

FIG. 22 is a bottom plan view of the multitool control mechanism in the intermediate position;

FIG. 23 is a sectional view of the mechanism taken along line 23-23 of FIG. 22 with the compressor omitted for clarity;

FIG. 24 is a graphical representation of a control mechanism for the multitool device;

FIG. 25 is a graph charting and button and clamp force on the y axis and button travel on the x axis;

FIG. 26 is a graphic representation of the different multitool actuation positions;

FIG. 27A is a perspective view of the multitool button in the IN position;

FIG. 27B is a top plan view of the multitool actuation system in the IN position with the handle shown in shadow line;

FIG. 27C is a side view of the multitool end effector in the IN position with the retractor head shown in shadow line;

FIGS. 28A-28C are, respectively, a perspective view of the multitool button in the OUT position, a top plan view of the multitool actuation system in the OUT position with the handle shown in shadow line, and a side view of the multitool end effector in the OUT position with the retractor head shown in shadow line;

FIGS. 29A-29C are, respectively, a perspective view of the multitool button in the HOME position, a top plan view of the multitool actuation system in the HOME position with the handle shown in shadow line, and a side view of the multitool end effector in the HOME position with the retractor head shown in shadow line;

FIGS. 30A-30C are, respectively, a perspective view of the multitool button in the OPEN position, a top plan view of the multitool actuation system in the OPEN position with the handle shown in shadow line, and a side view of the multitool end effector in the OPEN position with the retractor head shown in shadow line;

FIGS. 31A-31C are, respectively, a perspective view of the multitool button in the CLAMP position, a top plan view of the multitool actuation system in the CLAMP position with the handle shown in shadow line, and a side view of the multitool end effector in the CLAMP position with the retractor head shown in shadow line;

FIGS. 32A-32C are, respectively, a perspective view of the multitool button in the CUT position, a top plan view of the multitool actuation system in the CUT position with the handle shown in shadow line, and a side view of the multitool end effector in the CUT position with the retractor head shown in shadow line; and

FIG. 33 is a rear plan view of the yoke with shadow lines depicting the yoke at different positions within the handle of the multitool.

### DETAILED DESCRIPTION OF THE PREFERRED EMBODIMENT

Although this invention is applicable to numerous and various types of tissue to be severed, it has been found particularly useful in the environment of severing vessels such as side branches of a blood vessel being harvested. Therefore, without limiting the applicability of the invention to severing vessels such as side branches of a blood vessel being harvested, the invention will be described in such environment. Furthermore, the surgical devices of the present invention are preferably configured as disposable devices, however, the surgical devices can also be configured as semi-reusable or reusable without departing from the scope or spirit of the present invention.

System

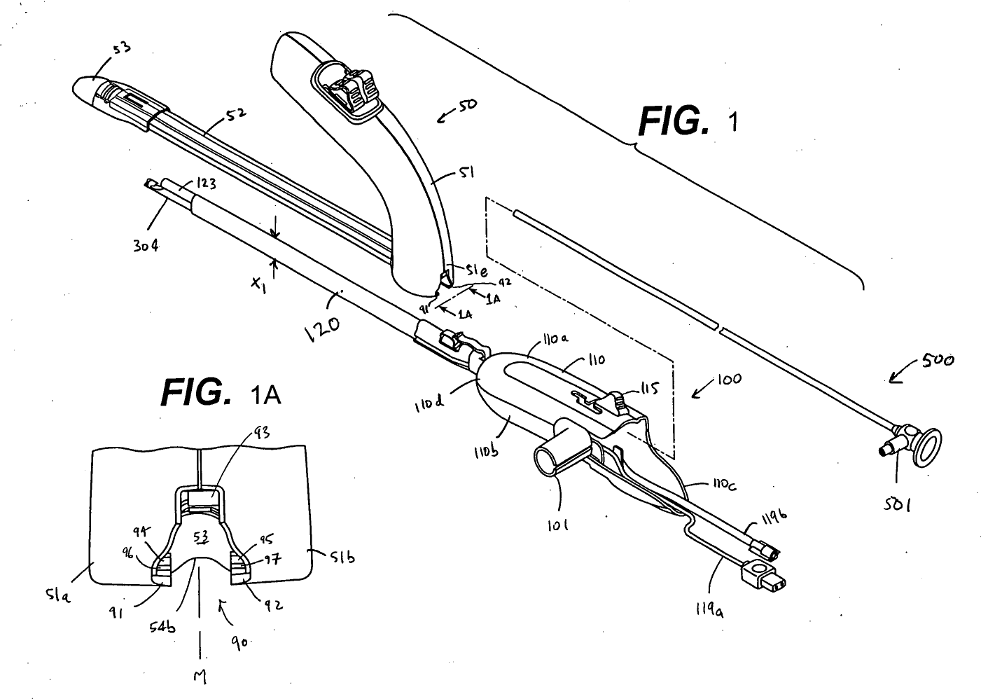

Referring to FIG. 1, a videoscopic endoscopic vein harvesting system is depicted, generally referred to as reference numeral 600. System 600 includes a retractor generally referred to as reference numeral 50, a multitool device generally referred to as reference numeral 100, an endoscope 500 slidable within multitool 100. A camera housing (not shown) can be matingly engaged with endoscope 500. In the perspective view of FIG. 1, retractor 50 and multitool 100 are shown in the undocked configuration, and endoscope 500 are shown as detached from multitool device 100.

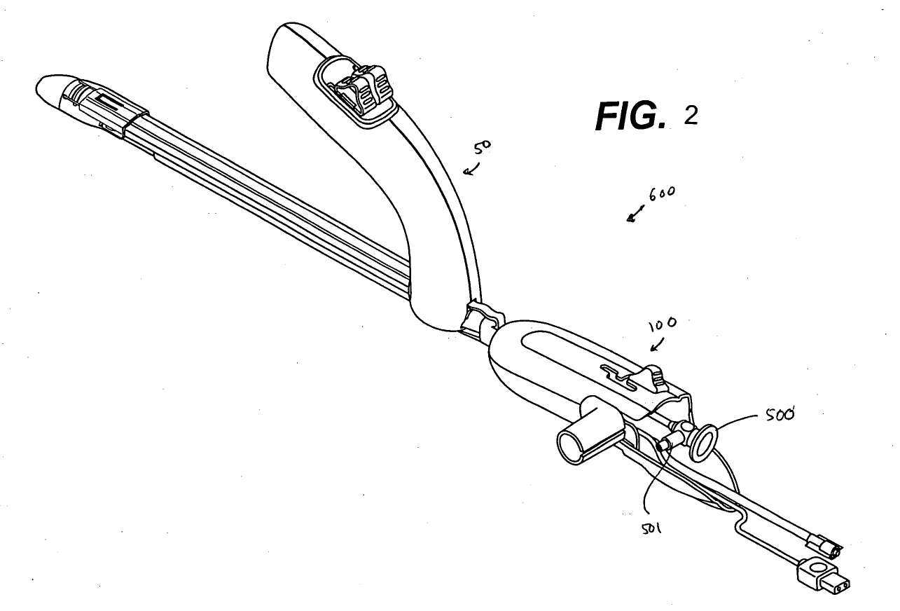

FIG. 2 depicts retractor 50 and multitool 100 in the docked configuration, and endoscope 500 engaged with multitool device 100. A description of the endoscope 500 and the camera housing are included in U.S. patent application Ser. No. 10/259,141, filed on Sep. 27, 2002, and entitled Portable, Reusable Visualization System, the contents of which are hereby incorporated by reference. When endoscope 500 is engaged with a handle 110 of multitool 100, a mating post 501 slides within shield 101. Mating post 501 typically heats up when endoscope 500 is being used and shield 101 serves to protect the user from being burned or distracted by the heat given off by mating post 501. Shield 101 is preferably attached to handle 110 of multitool 100, may be made of rubber or any thermoplastic elastomer, and preferably has a slit 101a to permit mating post 501 to easily slide within sleeve 101.

Retractor 50 and multitool 100 are described in some detail below as are the details of how and in what manner retractor 50 and multitool 100 are releasably attached or docked to one another.

Retractor

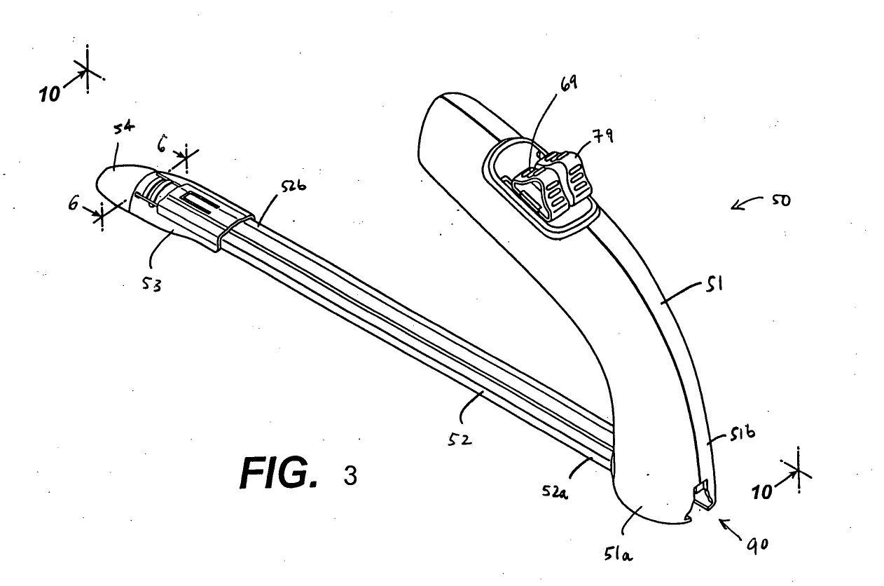

Referring to FIG. 3, a retractor, generally referred to by reference number 50, is depicted. Retractor 50 includes a handle 51, also serving as, and alternatively referred to as a housing, a shaft 52 extending distally from handle 51, and a working head 53 attached to the distal end of shaft 52.

Retractor 50 is typically used with an endoscope attached to or inserted through handle 51 and beneath shaft 52 so that an operator may view working space created by working head 53. In a preferred embodiment, retractor 50 is used in conjunction with a multitool instrument, more fully described in related U.S. patent application Ser. No. ______ (Attorney Docket No. ETH-5101), filed on the date of this application, and hereby incorporated by reference. U.S. Pat. No. 5,928,138 discloses how devices may be used with other instruments for dissecting and harvesting a vein, the disclosure of which is hereby incorporated by reference.

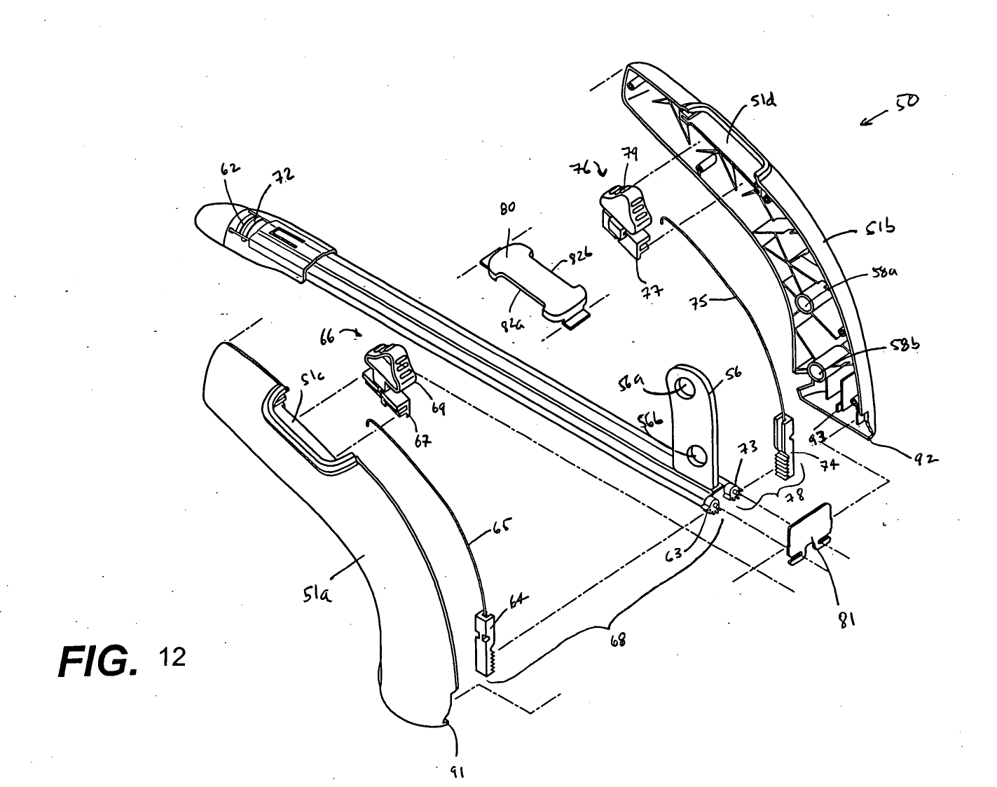

Retractor 50 may include a dock port 90 that releasably mates with a dock 140 of a multitool instrument 100 (FIG. 1) such that retractor 50 and multitool instrument 100 can be used together. Dock port 90 is preferably formed as part of handle 51. Referring to FIGS. 3 and 12, handle 51 is generally fabricated from a medical grade plastic and is preferably formed in a “clamshell” design having first and second halves 51a, 51b. The clamshell design allows for easy assembly of the internal components. The halves 51a, 52b are fixed together by any means known in the art, such as by a press fit, or with a medical grade epoxy or adhesive, or by ultrasonic welding or by mechanical means, such as by screws, or by any combination of the above.

As best shown in FIGS. 1 and 1A, dock port 90 is formed in handle 51 of retractor 50. Dock port 90 includes rails 91 and 92 that project inwardly from handle halves 51a and 51b, respectively, and extend longitudinally in a direction substantially parallel to shaft 52 of retractor 50 from a proximal end 51e to a distal end 51f of handle 51. Halves 51a, 51b are attached at a joint that extends generally along a medial plane M. Projections 94 and 95 project upwardly from the surface of rails 91 and 92, respectively, at a position near distal end 51f of handle 51. Slots 96 and 97 are formed in projections 94 and 95, respectively. Dock port 90 can also include a rib 93 that extends inwardly from handle half 51b at a position between proximal end 51e and distal end 51f of handle 51.

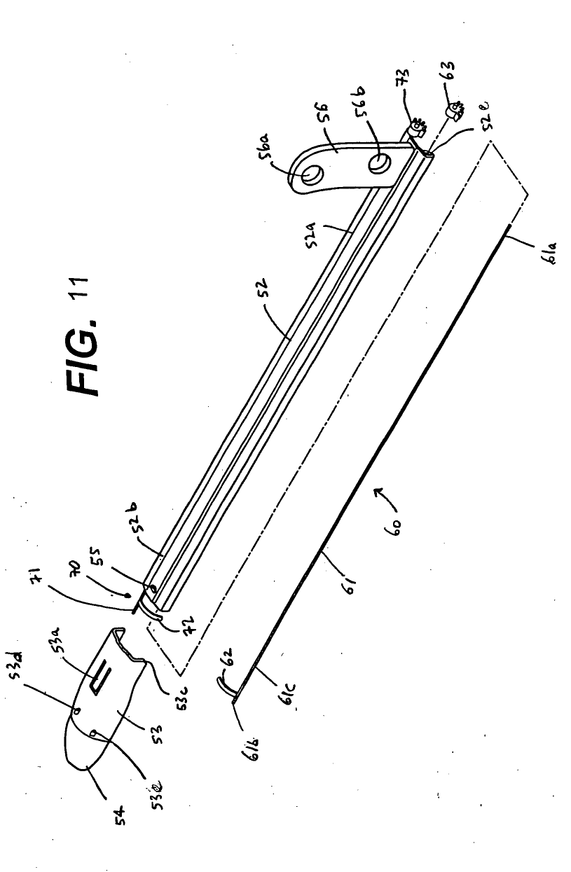

Referring to FIGS. 3 and 11, shaft 52 is fabricated from a medical grade resilient material, such as stainless steel. A proximal end 52a of shaft 52 is attached to a member 56, which extends upwardly from proximal end 52a. Member 56 may have openings 56a, 56b to facilitate attachment to handle 51 by any means known in the art, such as a press fit or a medical grade epoxy or adhesive or by heat-staking. Preferably, openings 56a and 56b of member 56 are sized to accommodate projections 58a, 58b (FIG. 12) that extend from each of halves 51a, 51b of handle 51 such that when halves 51a and 51b are brought together, the pairs of projections 58a and 58b capture member 56 by extending through openings 56a, 56b. A distal end 52b includes an opening 55 that is dimensioned to mate with a portion 53a of working head 53. Opening 55 is preferably formed by removing material from a cross-sectional portion of shaft 52. The removal of material to form opening 55 can be done by conventional machining or punching processes known in the art. Portion 53a of working head 53 is affixed to shaft 52 by any means known in the art, such as by a press fit and/or with a medical grade epoxy or adhesive. Shaft 52 is preferably shaped to form channels 52d and 52e (FIG. 5A) along a portion of the longitudinal length of shaft 52.

Working head 53 is useful for grossly dissecting tissue away from a vessel, such as the saphenous vein, when introduced through an incision in tissue, and creating a working space to permit the separation of the vessel from the surrounding tissue during EVH. Working head 53 is preferably made of a medical grade, injection-moldable plastic, such as polycarbonate, and is optionally clear for endoscopic viewing of tissue both inside and adjacent to working head 53. As is shown in FIG. 5A, working head 53 is preferably symmetrically shaped about a medial plane M and is generally concave.

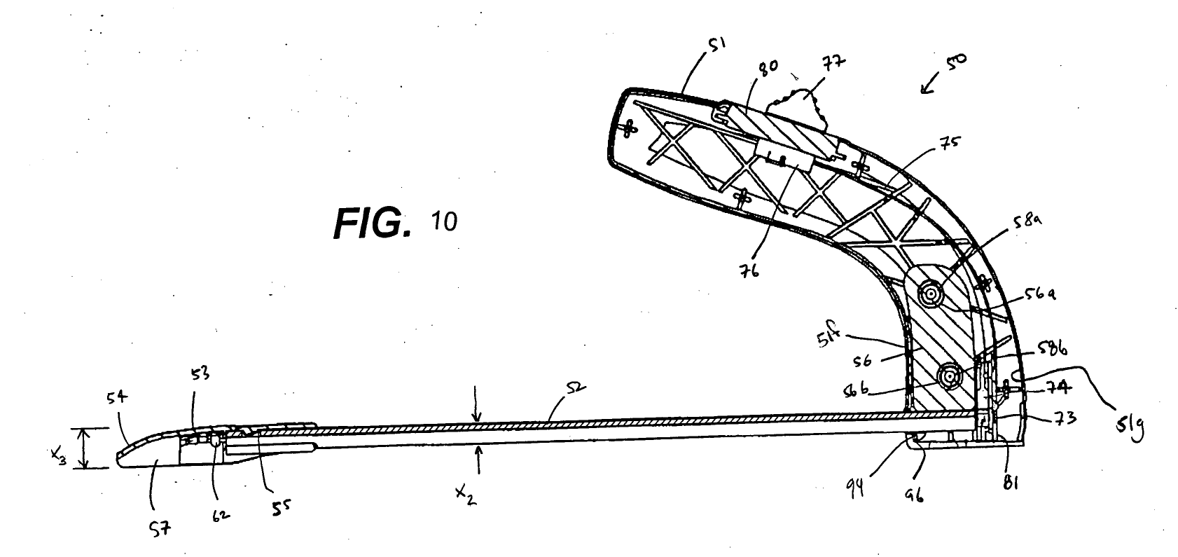

Referring to FIGS. 9 and 11, working head 53 tapers to a distal end 54 having a leading edge 54a so that an operator can easily use working head 53 to separate tissue layers and isolate a vessel from surrounding tissues. As is shown in FIG. 5A, working head 53 may have a notch 54b in leading edge 54a to provide for better visualization and management of anterior side branches. Working head 53 includes an outer surface 53b that terminates at a peripheral edge 53c. Working space 57 is defined as the area between the tissue overlying the blood vessel and the tissue underlying the blood vessel separated by working head 53. Working head 53 also includes recesses 53d and 53e spaced apart laterally from one another and substantially aligned with channels 52d and 52e, respectively, of shaft 52.

Working head 53 may have a spoon-shaped configuration, or it may consist of a bridge that extends for a portion or the full length of shaft 52, such as those depicted in U.S. Pat. No. 6,080,102, the disclosure of which is incorporate by reference. For example, working head 53 may consist of a tube having a semi-circular or a rhomboidal cross section when viewed axially. Such tubes may be entirely enclosed or have windows created therein. Working head may be slidable or fixed relative to shaft 52. In short, working head 53 can be any shape that defines a working space 57 that facilitates the introduction of instruments into working space 57 in order to perform various steps of a surgical procedure.

Referring generally to FIG. 11, retractor 50 also includes a vessel retractor system for manipulating a vessel proximate working space 57 during EVH by repositioning it within the operating field. In a preferred embodiment, the vessel retracting system includes a first manipulator 60, a first actuation system 68 (FIG. 12), a second manipulator 70 and a second actuation system 78. While the preferred system includes a first and second retractor, retractor 50 can include one or more retractors. In a preferred embodiment, retractor 50 includes a first manipulator 60 and a second manipulator 70, each disposed at least partially within working space 57. First manipulator 60 includes a first rod 61 having a proximal end 61a, a distal end 61b and a distal portion 61c, and a first paddle 62 extending from the distal portion 61c. First rod 61 is preferably made from stainless steel wire having a diameter approximately in the range of 0.025 inch to 0.075 inches, but most preferably 0.050 inches. A portion of rod 61 is disposed within channel 52d of shaft 52 with distal portion 61b extending beyond distal end 52b of shaft 52 and within working space 57. Distal end 61b is disposed within recess 53d of working head 53. Channel 52d and recess 53d are configured to retain a portion of rod 61, while permitting rod 61 to rotate freely within channel 52d and recess 53d. First paddle 62 is preferably attached to first rod 61 by laser welding, but could be attached by any means known to one skilled in the art.

Similarly, second manipulator 70 includes a second rod 71 having a proximal end 71a, a distal end 71b and a distal portion 71c, each of which are not shown in the figures, but are similar in form and function to the corresponding elements 61a, 61b and 61c of first manipulator 61. Manipulator 70 also includes a second paddle 72 extending from the distal portion 71c. Second rod 71 is preferably made from stainless steel wire having a diameter approximately in the range of 0.025 inch to 0.075 inches, but most preferably 0.050 inches. A portion of second rod 71 is disposed partially within channel 52e of shaft 52 with distal portion 71b extending beyond distal end 52b of shaft 52 and within working space 57. Distal end 71b is disposed within recess 53e of working head 53. Channel 52e and recess 53e are configured to retain a portion of second rod 71, while permitting second rod 71 to rotate freely within channel 52e and recess 53e. Second paddle 72 is attached to second rod 71 by laser welding, but could be attached by any means known to one skilled in the art.

Referring to FIG. 3, first paddle 62 and second paddle 72 are positioned offset distally from one another so as that one paddle does not to interfere with the other paddle's motion. Thus, first paddle 62 extends from first rod 61 at a location distal to the location where second paddle 72 extends from second rod 71. As such, first paddle 62 is retained within working head 53 at a location distal in a longitudinal direction to second paddle 72. Of course, either paddle could be configured in this way. In addition, first rod 61 and second rod 71 are offset from one another relative to the medial plane M of working head 53.

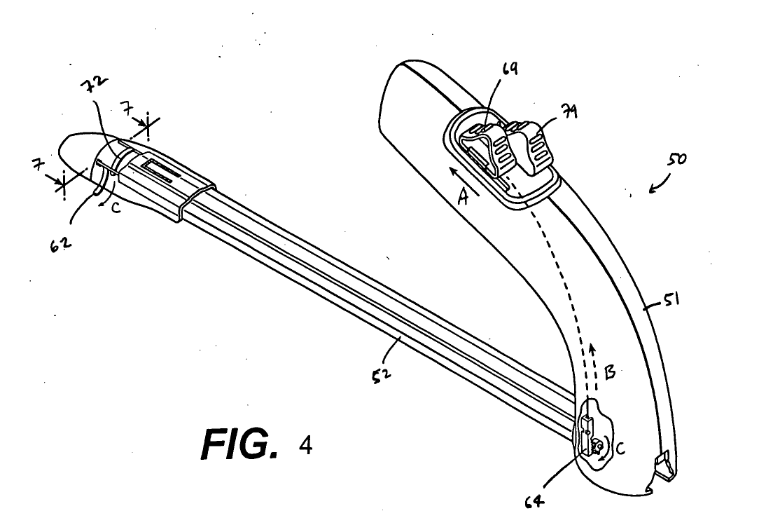

Referring now to FIGS. 4, 10 and 12, retractor 50 includes first actuation system 68 for moving paddle 62 between the retracted or stowed position and the extended position. In addition, the retractor 50 includes second actuation system 78 for moving paddle 72 between the retracted position and the extended position. The first actuation system is actuated by moving a first actuator 66 movably disposed in handle 52. First actuator 66 is preferably slidably disposed in handle 52 and operably connected to first paddle 62, such that moving first actuator 66a predetermined distance rotates first paddle 62 between the retracted and extended positions. Similarly, the second actuation system is actuated by moving a second actuator 76 movably disposed in handle 52. Second actuator 76 is preferably slidably disposed in handle 52 and operably connected to second paddle 72, such that moving second actuator 76a predetermined distance rotates second paddle 72 between the retracted and extended positions.

In a preferred embodiment, first actuator 66 of first actuation system 68 is operably attached to first paddle 62 so as to translate a linear motion to a rotational motion. First actuator 66 includes a first button 69 that the user moves to generate rotation of first paddle 62. First actuator 66 preferably also includes a slide 67 either integral with or separably attached to first button 69. First slide 67 is configured to retain one end of a wire 65 and to slidably ride in a slot 82a formed by lip 51c of handle 51 and a spacer 80. First wire 65 is connected at a distal end to first slide 67 and at a proximal end to a first rack 64. First rack 64, in turn is matingly engaged with a first pinion 63, which is preferably attached on one side to proximal end 61a of first rod 61 and rotates in a slot formed by backplate 81 and handle half 51a. Similarly, second actuator 76 of second actuation system 78 is operably attached to second paddle 72 so as to translate a linear motion to a rotational motion. Second actuator 76 includes a second button 79 that the user moves to generate rotation of second paddle 72. Second actuator 76 preferably also includes a slide 77 either integral with or separably attached to second button 79. Second slide 77 is configured to retain one end of a wire 75 and to slidably ride in a slot 82b formed by lip 51c of handle 51 and a spacer 80. Second wire 75 is connected at a distal end to second slide 77 and at a proximal end to a second rack 74. Second rack 74, in turn is matingly engaged with a second pinion 73, which is preferably attached on one side to proximal end 71a of second rod 71 and rotates in a slot formed by backplate 81 and handle half 51b.

Referring to FIG. 12, in a preferred embodiment, first and second racks 64, 74, first and second pinions 63, 73, and backplate 81 are all disposed within handle 51. Actuators 66, 76, racks 64, 74, pinions 63, 73 and spacer 80 are all preferably formed of a medical grade, injection moldable plastic, such as glass-filled nylon. Wires 65 and 75 are formed of a relatively flexible metal, such as stainless steel, and preferably range from 0.02 to 0.04 inches in diameter, and most preferably, is approximately 0.03 inches in diameter. Backplate 81 is preferably formed of stamped stainless steel.

Referring to FIG. 3, first button 69 and second button 79 are shown in their most proximal position, or the position closest to the operator's hand, within slots 82a and 82b. In this position, paddles 62 and 72 are retained within working head 53 in their stowed or retracted position. Referring to FIG. 4, displacement of first button 69 distally (or away from the operator's hand), in a direction depicted by arrow A, causes first wire 65 to move upwardly and distally (shown by broken arrow B), which in turn causes the first rack 64 to move upwardly. The motion of first rack 64 in turn causes first pinion 63 to rotate in the clockwise direction depicted as arrow C. As pinion 63 is attached to rod 61, rotation of first pinion 63 causes first paddle 62 to also rotate in the clockwise direction. Similarly, referring to FIG. 5, moving second button 79 distally in a direction depicted by arrow D causes second wire 75 to move upwardly and distally, which in turn causes second rack 74 to move upwardly, causing second pinion 73 and second paddle 72 to rotate in a counter-clockwise direction shown by arrow E.

First button 69 and second button 79 are positioned side by side such that a user that grasps retractor 50 with one hand, may actuate either or both buttons by using a thumb or finger. Thus, the user can manually retract tissue to form working space 57 and retract the vessel being harvested by using retractor 50, without the need for a separate instrument. Further, because retractor 50 includes first paddle 62 on one side of the medial plane M of retractor 50 and second paddle 72 on the other side of the medial plane of retractor 50, the user may move the vessel to one side away from the medial plane of retractor 50 using first paddle 62 or the other side away from the medial plane of retractor 50 using second paddle 72, without the need to reposition or rotate retractor 50. Thus, in the event the user would like to transect a side branch on the right side of vessel, the user can use first paddle 62 to manipulate the vessel away from the side branch, and, similarly, where the user would like to transect a side branch on the left side of vessel, the user can use second paddle 72 to manipulate the vessel away from the side branch.

While the preferred embodiment depicts a first and second actuation system 68, 78, it is contemplated that first retractor and second retractor could be actuated using one actuation system. For example, rather than having buttons that go up and down, a single button can be toggled left or right to engage slide 67 or slide 77 depending upon which manipulator the user wanted to actuate. As a result, other than the toggle motion, the remainder of the actuation mechanism would work similarly to the described device; i.e., slides 67, 77 could move wires 65, 75 and racks 64, 74 to act upon pinions 63, 73 and manipulators 60, 70.

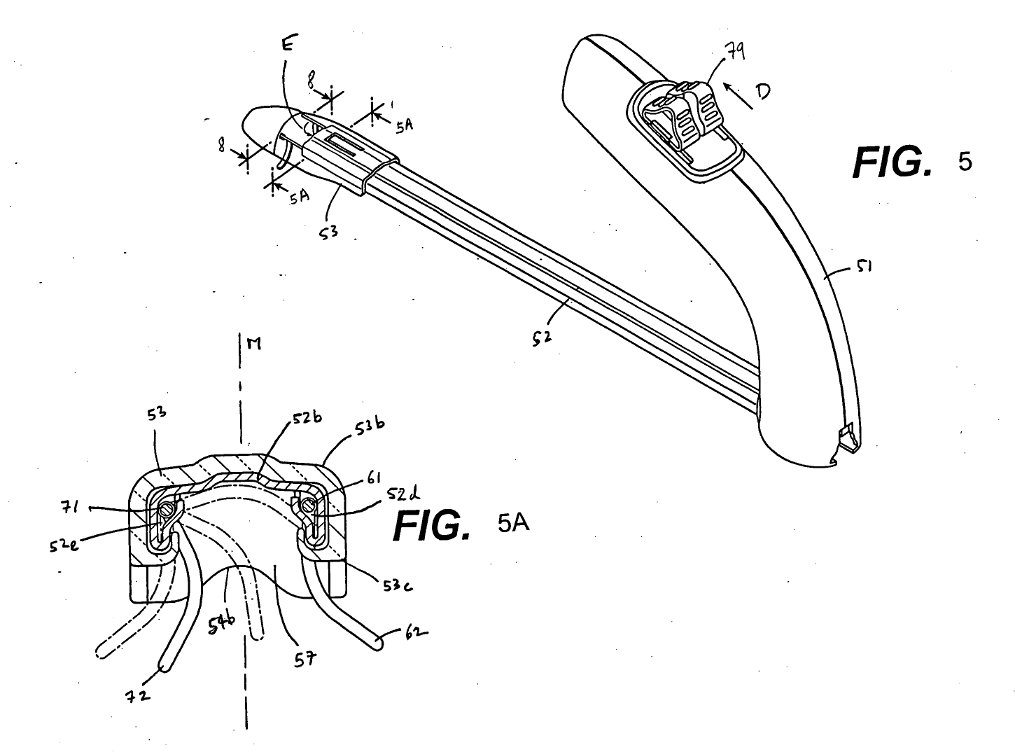

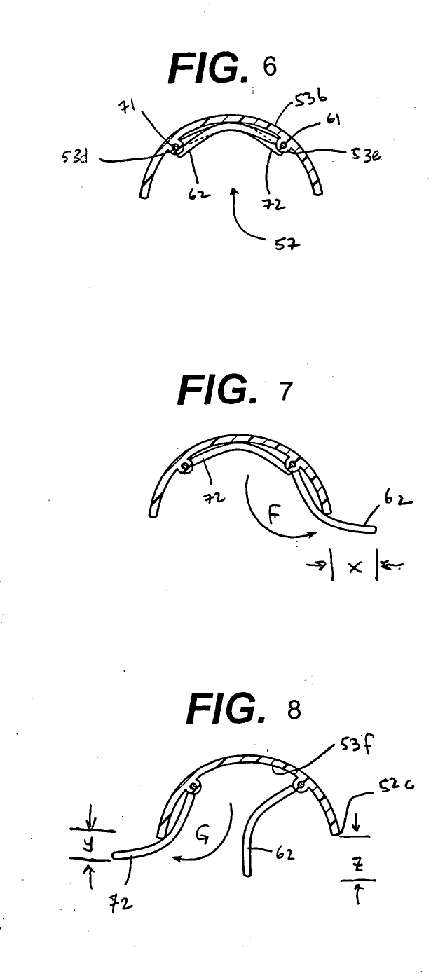

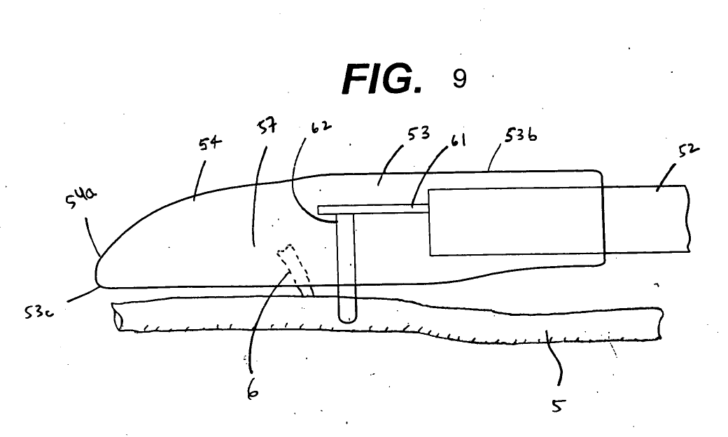

Referring to FIGS. 6-9, the details of the distal end of retractor 50 are shown. Referring to FIG. 6, first paddle 62 and second paddle 72 are shown in their stowed or retracted position. First paddle 62 and second paddle 72 are positioned to nest longitudinally in a side-by-side configuration close to a portion of the interior surface 53f of working head 53. In the stowed position, first paddle 62 and second paddle 72 are preferably shaped to substantially minimize the amount of working space obstructed by the paddles themselves. Preferably, as is shown in FIG. 7, first paddle 62 may rotate about the pivot point defined in recess 53d through an arc F of approximately 100 to 140 degrees, but most preferably 120 degrees. Similarly, as is shown in FIG. 8, second paddle 72 may rotate about the pivot point defined in recess 53e through an arc G of approximately 100 to 140 degrees, but most preferably 120 degrees. In each case, however, it is contemplated that the angle of rotation could be greater or smaller depending upon the location of recesses 53d, 53e and the curvature of working head 53.

As is shown in FIGS. 7 and 9, first paddle 62 extends below peripheral edge 53c defined by working head 53 when first paddle 62 is in the extended position. Preferably, first paddle 62 has a curved portion that forms a concave surface that faces away from working head 53 when in the extended position. In a preferred embodiment, when in the fully extended position, paddles 62 and 72 extend a distance X of approximately 0.10 inches to 0.25 inches medially outwardly (FIG. 6) from working head 53, but most preferably approximately 0.15 inches, and downwardly (FIG. 9) from working head 53a distance Y of approximately 0.15 inches to 0.35 inches, but most preferably approximately 0.20 inches. When paddle 62 or 72 is extended below peripheral edge 53c normal to pivot point 53d, 53e, the tip of paddle 62, 72 (FIG. 8) preferably extends a distance Z of approximately 0.15 inches to 0.35 inches below edge 52c, but most preferably approximately 0.25 inches. The length of the paddles is preferably configured to be long enough to manipulate a vessel to a position that does not interfere with the working space, but short enough so as not to be prevented from rotating by the layer of tissue at the bottom of the working space when the paddles are actuated.

Multitool

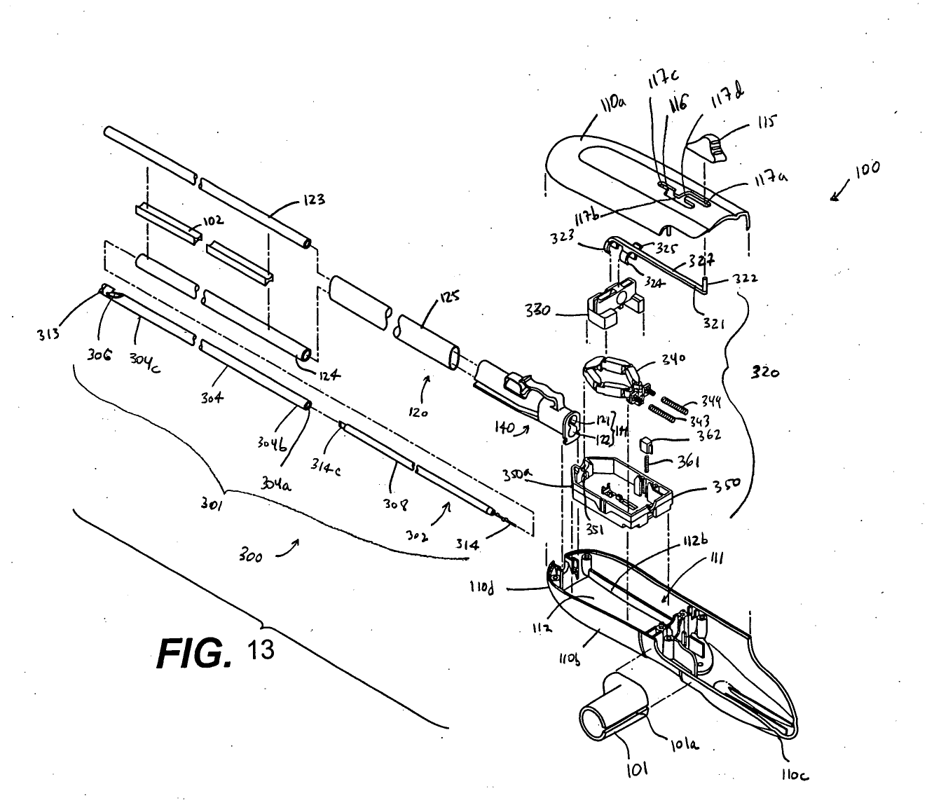

Referring now to FIGS. 1 and 13, multitool device 100 is depicted. Multitool 100 includes a surgical device 300 that is slidable within tube 124, and includes a shaft 304 having an opening 306 at a distal end configured to capture tissue. Surgical device 300 includes an anvil assembly 302 slidable within shaft 304 for clamping tissue captured within opening 306 and a cutting blade 314 slidable within shaft 304 for cutting the captured tissue. Surgical device 300 also includes at least one electrode for providing RF energy to desiccate the captured tissue.

Multitool device 100 preferably includes a handle 110, also serving as, and alternatively referred to as a housing. Handle 110 has a button 115 slidably disposed therein, and a cannula 120 that projects from handle 110. Handle 110, as with handle 51 of retractor 50, is fabricated from a medical grade thermoplastic and is preferably formed in a “clamshell” design having first and second halves 10a, 10b. The clamshell design allows for easy assembly of the internal components. The halves 110a, 110b are fixed together by any means known in the art, such as by a press fit, or with a medical grade epoxy or adhesive, or by ultrasonic welding or by mechanical means, such as by screws, or by any combination of the above. Handle 110 has a proximal end 110c and a distal end 110d. Proximal end 110c is configured to mate with a camera portion (not shown), which is described in detail in U.S. patent application Ser. No. 10/259,141, filed on Sep. 27, 2002, and entitled Portable, Reusable Visualization System, the contents of which are hereby incorporated by reference.

Handle halve 10a has a slot 116 formed therein. Slot 116 has a first track 117a, a second track 117b that communicates with first track 117a, and a third track 117c that communicates with second track 117b. First track 117a is preferably located on one side of a medial plane M and extends longitudinally toward the distal end of shaft 304. The medial plane M is centered along the longitudinal axis of tubes 123, 124. Second track 117b also extends longitudinally, is preferably located on the other side of medial axis M and is connected to first track 117a by a fourth track 117d that extends substantially normal to first track 117a and second track 117b. Third track 117c begins at the distal end of second track 117b and extends longitudinally along a line substantially along medial axis M.

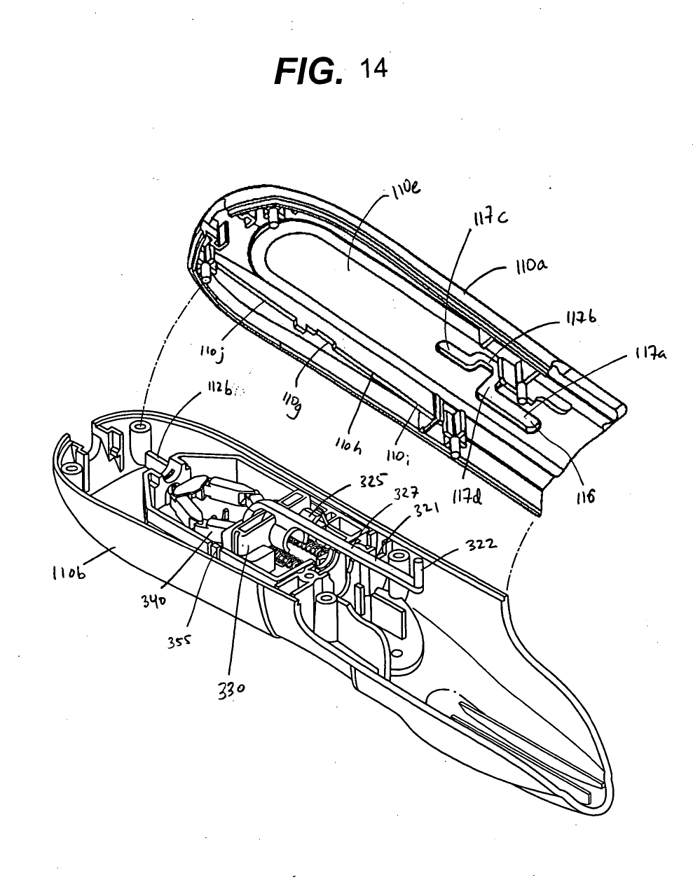

Referring to FIG. 14, the underside 110e of handle half 110a is depicted. A ramp 110h extends from underside 110e and tapers from a first height 110i to a second shorter height 110j. Ramp 10h has a notch 110g at a location corresponding to the location of tab 325 of yoke 321 (described below) when sled 350 is at the distal position.

Preferably, multitool device 100 has a tube 119b for providing a fluid for defogging or clearing endoscope 500. Tube 119b has a proximal end which is in fluid communication with a fluid source, and a distal end that communicates with tube 124, thereby providing a fluid, such as carbon dioxide, to clear endoscope 500 when it is disposed within tube 124.

Cannula 120 of multitool device 100 preferably has two lumens, but may have additional lumens. In the preferred embodiment, a first lumen 121 is sized to accommodate an endoscope, and a second lumen 122 is sized to accommodate a tool such as a surgical device 300. Cannula 120 may be formed of a metal, or of a hard plastic or of a combination of metal and plastic. In a preferred embodiment, first and second lumens 121, 122 of cannula 120 are formed by separate tubes 123, 124 that are spaced with respect to one another by a spacer 102 that extends for a desired length between tubes 123, 124. Tubes 123, 124 are alternatively referred to as shafts. Tubes 123, 124 provide rigidity as they are preferably formed of a metal, however, tubes 123, 124 are not essential to the invention as long as the endoscope and surgical device 300 are fixed with respect to each other and multitool device 100 is of sufficient rigidity.

First tube 123 is dimensioned to house an endoscope (not shown) that is passed through handle 110 from a proximal end to the distal end and through tube 123 such that it extends distally from the distal end of tube 123. Tubes 123, 124 have a length of length of approximately 10.5 inches, and a diameter of about 0.25 inches. First and second tubes 123, 124 are preferably fixed with respect to one another by an outer sheath 125 that extends longitudinally along a substantial portion of tubes 123, 124. Sheath 125 is preferably heat shrunk around tubes 123, 124.

As discussed above, retractor 50 may include a dock port 90 to mate with a dock 140 of a multitool instrument 100 so retractor 50 and multitool instrument 100 can be used together. Dock 140 and dock port 90 include at least one docking feature that secures dock 140 and dock port 90. One skilled in the art can devise numerous docking features, among which would be a latch, a rail and slot configuration, a luer lock. It should be understood that multitool instrument 100 may include one or more different surgical devices and does not necessarily need to include an endoscope. For example, an endoscope can be supplied with retractor 50.

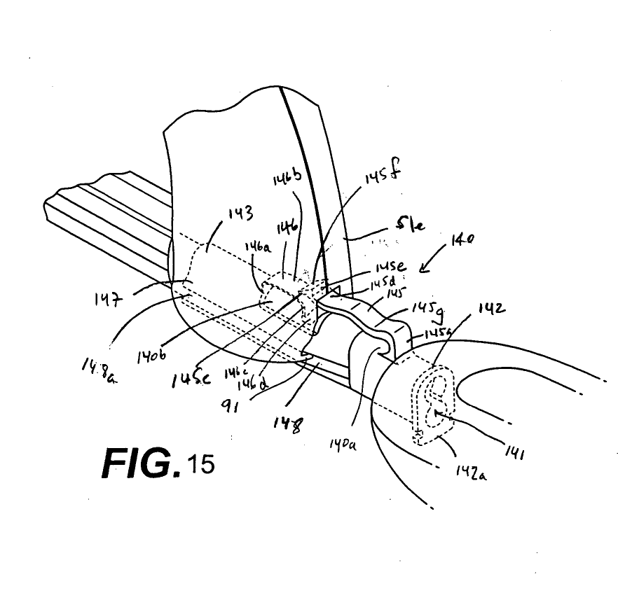

Returning to the description of multitool device 100 and referring to FIGS. 13 and 15, device 100 also includes a dock 140 preferably located between the proximal end of tubes 123, 124, and handle 110. Dock 140 is preferably formed of a hard plastic that is injection molded to form features that mate and interact with dock port 90. Dock 140 preferably includes a passageway 141 that accommodates lumens 121, 122, a proximal end 142 having a projection 142a that is captured within joined handle halves 10a and 10b of multitool handle 110, and a distal end 143 that is configured to be disposed within dock port 90 of retractor 50 when retractor 50 and multitool device 100 are in the docked configuration.

Dock 140 preferably includes projections 147 on either side (only one of which is depicted in FIG. 15). Projections 147 each have a slot 148 formed therein at a location preferably substantially aligned with the upper edge of second lumen 122 or second tube 124 when dock 140 and dock port 90 are in the docked configuration. Projections 147 and slots 148 are preferably formed in dock 140 by injection molding and are configured to slidably accept rails 91 and 92, respectively, of retractor 50. Slots 148 each have at a distal end thereof a mouth 148a that is slightly larger than the remainder of slot 148 to permit rails 91 and 92 to be more easily slid into slots 148. Preferably slots 148 are wider than the width of rails 91, 92 such that there is some play between slots 148 and rails 91, 92. Mouths 148a and the play between slots 148 and rails 91, 92 permit multitool device 100 to be pivoted downwardly with respect to retractor 50. To further secure multitool device 100 to retractor 50, dock 140 may include ridges 147a (one on either side of dock 140) that are configured to be accepted in slots 96 and 97 of dock port 90.

Referring to FIG. 15, dock 140 also includes a latch 145, and a leaf spring 146 positioned distally to latch 145. Latch 145 projects upwardly from an upper surface 140a to form a leg 145a, and extends substantially longitudinally at a location spaced apart from upper surface 140a to form an arm 145b having a distal free end 145c. Arm 145b includes a distal projection 145d at a distal end that has a face 145e, that extends substantially parallel to leg 145a, and a ramp 145f that angles downwardly toward upper surface 140a. Leaf spring 146 projects upwardly from upper surface 140a distal a window 140b in upper surface 140a, and includes a first leg 146a, a beam 146b that extends proximally from first leg 146a, and a second leg 146d that extends from the proximal end of beam 146b. Second leg 146d preferably includes a seat 146c that is formed as an arc that is configured to ride on the outer surface of tube 123 when beam 146b is deflected.

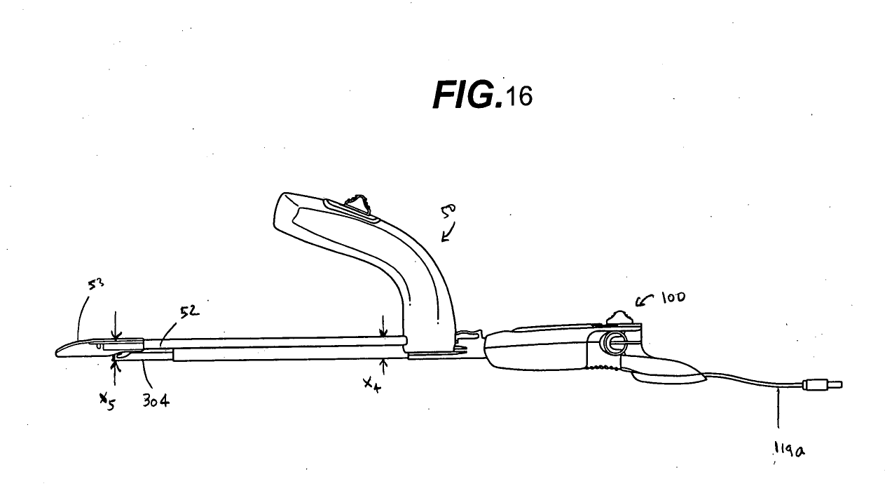

FIG. 16 depicts a plan view of retractor 50 and multitool 100 in the docked configuration. Dock 140 and port 90 are configured such that the end effector of surgical device 300 of multitool 100 is positioned within working space 57 when dock 140 and port 90 are in the docked configuration. Multitool 100 and surgical device 300 are described in detail in related U.S. patent application Ser. No. ______ (Attorney Docket No. ETH-5101), filed on the date of this application and assigned to Ethicon, Inc, and hereby incorporated by reference.

In the docked configuration, the distal end of multitool 100 is disposed within working space 57 of retractor 50 and advantageously minimizes the stack-up height of the docked instruments. Referring to FIG. 1, the height x₁ of multitool 100 is approximately 0.53 inches. Referring to FIG. 10, the height x₂ of shaft 52 of retractor 50 is approximately 0.28 inches and the height x₃ measured from the top of working head to the lower edge of peripheral edge 53c is approximately 0.53 inches. Referring to FIG. 16, the height x₄ of retractor 50 and multitool 100 at a location where the docked devices enters an incision is approximately 0.66 inches, and the height x₅ measured from the top of working head 53 to the underside of distal end 304c of shaft 304 of multitool 100 is approximately 0.57 inches. Thus, in the docked configuration shaft 304 of multitool 100 is slightly biased toward the underside of working head 53 as the stack-up height decreases from 0.66 inches at the typical point of insertion to 0.57 inches at the most distal location of the docked devices. As a result, retractor 50 when docked with multitool 100 only creates an additional stack up height of approximately 0.04 inches at the distal-most point. This arrangement provides the user with sufficient operative space, while minimizing the amount of tissue manipulation, and permits easy movement of the multitool 100 through the operative space, whether in a docked or undocked configuration.

Referring to FIGS. 1 and 15, when a user wishes to place multitool device 100 in the docked configuration with retractor 50, the user positions retractor 50 over the upper surface of tube 123 (or sheath 125 that covers tube 123), and aligns port 90 with dock 140. The user slides retractor shaft 52 over tube 123 such that rails 91, 92 enter mouths 148a of slots 148 until proximal end 51e of handle 51 contacts ramp 145f. As the proximal end 51e rides up ramp 145f, latch 145 deflects toward upper surface 140a. When proximal end 51e clears ramp 145f, face 145e resides within handle 51 and abuts an inner surface 51g (FIG. 10) of handle 51, and projections 147 of dock 140 reside within slots 96 and 97 of port 90. In this manner, longitudinal or axial movement of multitool 100 with respect to retractor 50 is prevented.

In addition, at this position, beam 146b pushes against rib 93 of retractor 50 thereby biasing the end effector or distal end of multitool 100 toward working head 53 of retractor 50. The user may, however, exert a spreading force on the handle 51 of retractor 50 and/or handle 110 of multitool 100 that can deform beam 146b such that seat 146c slides proximally on upper surface of tube 123 thereby temporarily overcoming the spring force of leaf spring 146 and permitting the distal end of multitool 100 to be deflected downwardly with respect to working head 53. In this manner, the user is provided a degree of freedom (DOF) for extra manipulation to, for example, to stow manipulators 62, 72 without having to undock retractor 50 from multitool 100. When hand pressure is removed by the user, the distal end of the multitool 100 is automatically biased upwards due to leaf spring 146.

To undock the multitool from retractor, the user presses downwardly on a concave surface 145g of latch 145 such that distal end 145c of latch 145 moves downwardly out of engagement with proximal end 51d of housing 51 thereby permitting the user to move retractor 50 distally with respect to multitool 100 to separate one from the other.

FIGS. 13 and 15 depict one embodiment of a docking arrangement. While dock 140 is shown with two slots 148, dock 140 does not necessarily require any slots or could use just one slot formed, for example, at the lower edge of dock 140, or more than two slots. Other arrangements can clearly be envisioned by those skilled in the art. For example, a fully rigid dock that eliminates all degrees of freedom; a dock that permits axial or longitudinal movement; a dock that permits axial rotation or radial movement of multitool 100; a detent dock, or any combination of the above. In addition, while port 90 is described as an element of retractor 50 and dock 140 is described as an element of multitool 100, those skilled in the art will understand that the reverse design will work just as well. That is, multitool 100 can include a port 90 and retractor 50 can include a dock 140.

Referring to FIGS. 1 and 13, surgical device 300 is depicted. Surgical device 300 includes a shaft 304, a tip 313 disposed at a distal end of shaft 304, an anvil 308 disposed at least partially within shaft 304, at least one electrode for cauterizing tissue, and a cutting blade 314 also disposed at least partially within shaft 304. Shaft 304 is preferably at least partially slidably disposed within tube 124. Shaft 304 has a first internal lumen 304a, a proximal end 304b and a distal end 304c. Shaft 304 is fabricated from a medical grade resilient material, such as stainless steel, and preferably is affixed at proximal end 304b to a sled 350 by any means known in the art such as by press fit or with an adhesive. Preferably, proximal end 304b is attached to distal end 350a of sled 350 within an opening 351 in distal end 350a.

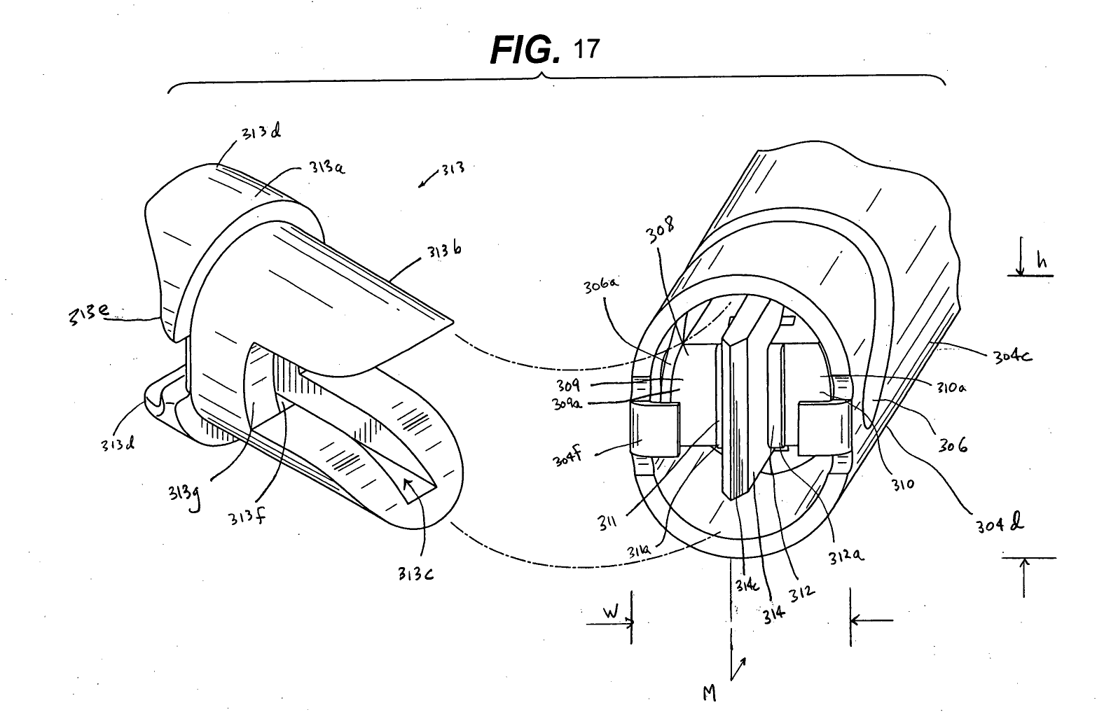

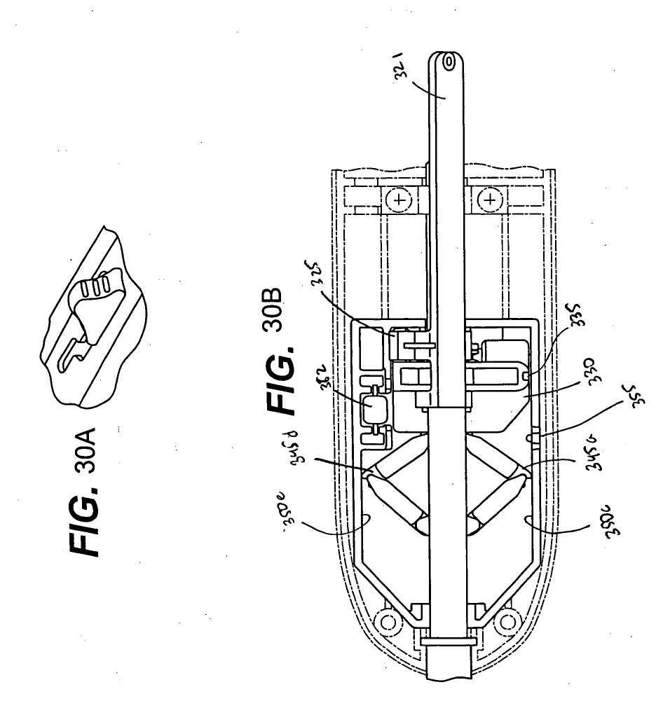

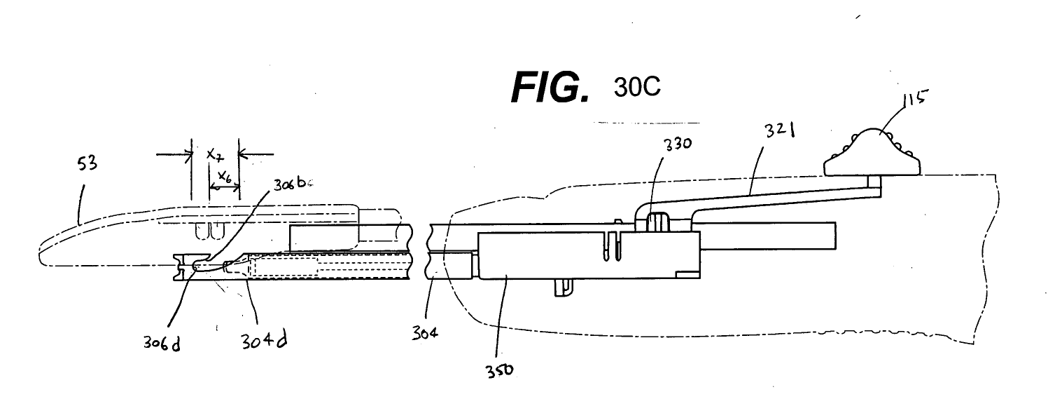

Shaft 304 has an opening 306 at a distal end 304c. Opening 306 is preferably formed by removing material from a cross-sectional portion of the shaft 304 such that opening 306 has a peripheral edge 306a defining the boundaries of opening 306. The removal of material to form opening 306 can be performed by conventional machining or punching processes known in the art. Referring to FIGS. 17 and 30C, shaft 304 has a distal segment 304d that is has an oblong cross section. In a preferred embodiment, the height h of distal segment 304d is approximately 5.5 mm and the width w of distal segment 304d is approximately 4.5 mm. The oblong cross section provides greater height to distal segment 304d, which permits opening 306 to be larger without the sacrificing structural integrity of distal segment 304d. Opening 306 may be configured to accommodate the largest size blood vessel possible for a given diameter of shaft 306. In a preferred embodiment, and referring to FIG. 30C, shaft 304 diameter is approximately 2 mm, and opening 306 has a mouth length x₆ of approximately 7 mm and an overall length x₇ of approximately 11 mm. The radius of a distal semicircular portion 306d of opening 306 is approximately 2 mm. This configuration permits blood vessels as great as 7 or 8 mm to be accepted within opening 306 due to the flexibility of blood vessels.

Referring to FIGS. 13 and 17, surgical device 300 also preferably includes a tip 313 disposed at the distal end 304c of shaft 304 for dissecting tissue. Tip 313 is shaped so that it can perform blunt dissection when needed and manipulate tissue. Tip 313 includes a distal portion 313a and a proximal portion 313b. When tip 313 is attached to shaft 304, distal portion 313a extends beyond distal end 304c of shaft 304, while proximal portion 313b is preferably substantially disposed within the hollow distal end 304c. Distal portion 313a of tip 313 preferably is c-shaped such that distal portion 313a has wide portions 313d and a narrowed portion 313e. Wide portions 313d serve to channel tissue distal of tip 313 toward cutting blade 314 when cutting blade 314 is exposed within distal portion 313a. Wide portions 313d also serve to limit the tissue exposed to cutting blade 314 and shield tissue from the sharp edges of cutting blade 314.

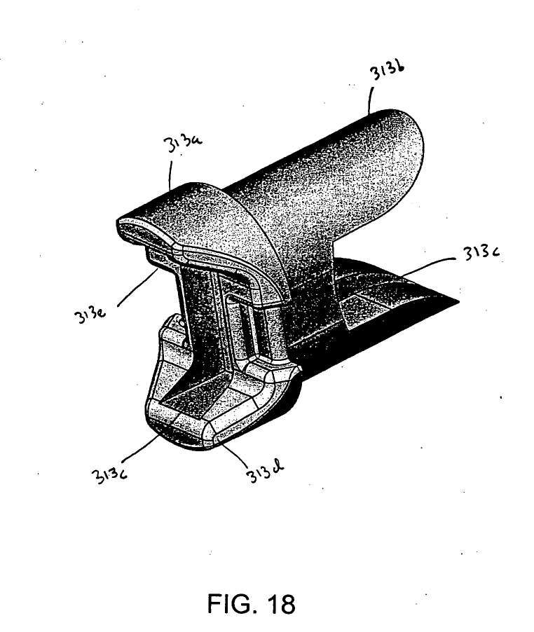

Referring to FIGS. 17 and 18, tip 313 is preferably separately formed from shaft 304 and attached to shaft 304 by any means known in the art such as by a press fit, medical grade epoxy, brazing or welding. In a preferred embodiment, tip 313 is attached by way of tabs 304f that extend distally from distal end 304c of shaft 304 prior to assembly with tip 313. Tabs 304f of shaft 304 are then bent, preferably over the narrowed portion 313e, during assembly to the position shown in FIG. 17 to retain tip 313 to distal end 304c of shaft 304. Tip 313 can also be integrally formed with shaft 304, however, such as by rolling distal edge 304c of shaft 304 into an appropriate shape. To maintain more consistent and robust tissue contact, proximal portion 313b of tip 313 is recessed from the distal end 304c of shaft 304 such that distal end 304c of shaft 304 contacts tissue captured within opening 306 without interference from proximal portion 313b.

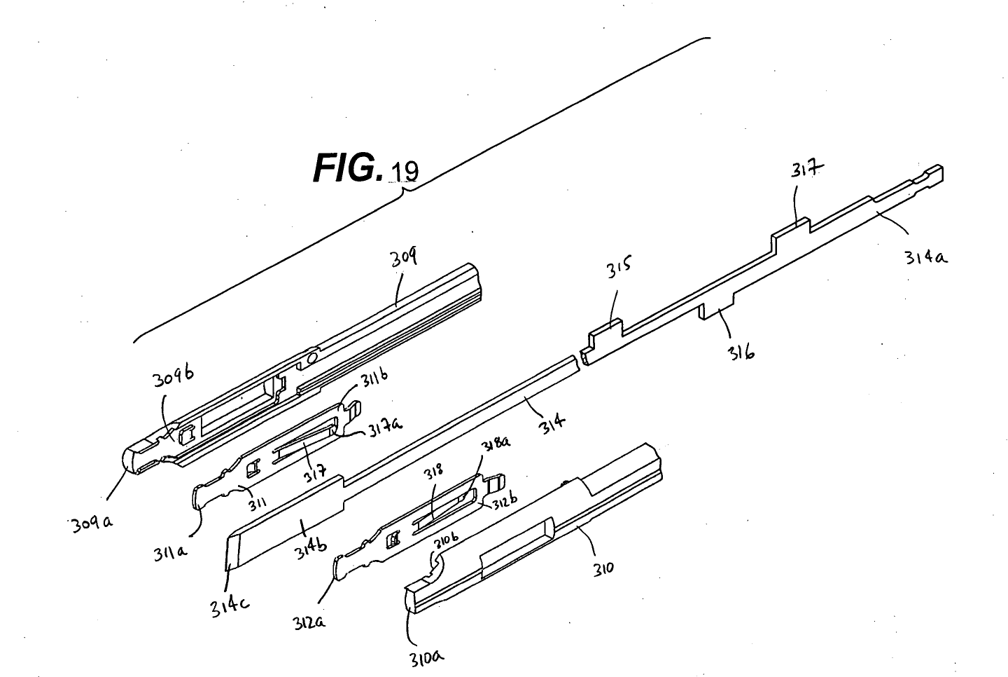

Referring now to FIGS. 17 and 19, surgical device 300 also includes cutting blade 314 slidingly disposed in opening 306 between open and closed positions. In a preferred embodiment, cutting blade 314 is slidable between a proximal position, an intermediate position, and a distal position. Cutting blade 314 preferably has a proximal end 314a having a first height, a distal end 314b having a second height, and a sharpened cutting edge 314c at distal end 314b. Cutting edge 314c of cutting blade 314 can be heat-treated to maintain a sharp edge. The distal height of cutting blade 314 (and distal end 314b) ranges from 0.10 inches to 0.20 inches, but preferably is approximately 0.15 inches. Cutting blade 314 narrows to proximal end 314a to a second height that is approximately 0.05 inches.

Cutting blade 314 preferably has a first flag 315, a second flag 316 and a third flag 317 that extend from proximal end 314a at spaced-apart locations. Preferably, second flag 316 extends in a direction opposite from first flag 315 and third flag 317 and acts as a stop to prevent further distal movement, when cutting blade is moved from a proximal position to a distal position. As is described in more detail below, first and third flags 315, 317 are engaged to respectively push cutting blade 314 distally and pull cutting blade 314 proximally, depending upon how the user actuates the device.

Proximal end 314a of cutting blade 314 is preferably disposed within handle 110 and is attached to a control mechanism described below. Proximal end 314a preferably slides within sled 350 of control mechanism 320. In its most proximal position, shown as OPEN position 740 (FIG. 30B), proximal end 314a may extend through opening 354 of sled 350 (FIG. 21). Preferably cutting blade 314 slides through distal end 340a of flexure mechanism 340 through a space defined by rods 345c and 345b and out distal end 340b of flexure mechanism 340 through a space between first and second posts 341, 342 and through channel 336 formed by compressor 330. At least a portion of cutting blade 314 may be wrapped in a dielectric insulator, such as a polymer.

Cutting blade 314 is preferably slidingly disposed within shaft 304. In the proximal or open position, cutting blade 314 does not substantially interfere with capturing tissue in opening 306. While in the intermediate or closed position, cutting blade 314 contacts and cuts the tissue captured between the clamping surface 308a and at least a portion of opening 306a. When cutting blade 314 is moved to its most distal position disposed within the contours of distal portion 313a of tip 313, it is preferably spring-biased such that when the user releases button 115, cutting edge 314c moves proximally to a more proximal position within distal portion 313a of tip 313.

Referring to FIG. 17, tip 313 preferably has a slot 313c formed therein for acceptance of at least cutting edge 314c of cutting blade 314. In the distal position, at least cutting edge 314c extends through slot 313c such that cutting edge 314c extends beyond narrowed portion 313e of tip 313.

Surgical device 300 includes at least one electrode provided on surgical device 300 for applying RF energy to the tissue captured in opening 306. As used herein, an electrode is any element capable of conducting electricity that is connected to an energy source. Preferably, surgical device 300 is configured to apply RF energy to cauterize the captured tissue and more preferably, surgical device 300 is further configured as a bipolar device. The preferable means for cauterization is given, however, by way of example only and not to limit the scope or spirit of the present invention. For instance, surgical device 300 can be used in a monopolar configuration in combination with a grounding plate as is known in the art. Furthermore, surgical device 300 can be configured to apply sonic energy to cauterize the captured tissue.

In the preferred bipolar configuration, the at least one electrode comprises first and second electrodes, each of a different polarity. In one embodiment, the first electrode comprises at least cutting edge 314a of cutting blade 314 and the second electrode comprises at least a portion of shaft 304. The at least a portion of shaft 304 comprises the edge 306a defining opening 306. Alternatively, the first electrode comprises at least the clamping surface of an anvil 308 (described below) and the second electrode comprises at least a portion of shaft 304.

To mitigate any thermal damage that may occur to surrounding (non-target) tissue due to the RF energy, the device is preferably designed to utilize offset-bipolar technology. Referring to FIGS. 17 and 19, for a more detailed view of the distal end of surgical device 300, preferably, the at least one electrode comprises a first electrode 311 and a second electrode 312 spaced from the first electrode 311, each having the same polarity. At least a portion of shaft 304 acts as a third electrode having the opposite polarity of first and second electrodes 311, 312. The first and second electrodes 311, 312 are preferably located close to the medial plane M of shaft 304. Shaft 304 is spaced apart from first and second electrodes 311, 312, such that electrodes 311, 312 and shaft 304 are offset from one another when tissue is captured within opening 306.

First and second electrodes 311, 312 are preferably elongate and are configured to be disposed at least partially within distal end 304c of shaft 304 on either side of cutting blade 314. First electrode 311 and second electrode 312 each have a distal portion 311a, 312a, that may extend beyond clamping surfaces 309a, 310a, respectively. Distal portions 311a, 312a of electrodes 311, 312 may also be flush with clamping surfaces 309a, 310a, or recessed within clamping surfaces 309a, 310a. In an embodiment where distal portions 311a, 312a extend beyond clamping surfaces 309a, 310a, tissue clamped between anvil assembly 302 (which includes electrodes 311, 312) and proximal portion 313b of tip 313 must navigate a tortuous path over distal portions 311a, 312a, which ensures that the captured tissue maintains good, robust electrical conduct with electrodes 311, 312. In addition, tip 313 includes recesses 313f (one shown in FIG. 17) formed in proximal portion 313b sized and configured to accept electrodes 309, 310 when tissue is clamped within opening 306 by anvil 308.

In addition to distal portions 311a, 312a, first and second electrode 311, 312 each have a proximal portion 311b, 312b, and each includes a spring 317, 318 that is biased toward the medial plane M of shaft 304. Preferably, springs 317, 318 are located at proximal portion 311b, 312b and are formed by removing material from electrodes 311, 312 such that a portion 317a, 318a of springs 317, 318 is biased toward medial plane M. Portions 317a, 318a maintain contact with cutting blade 314 at least when cutting blade 314 is in its most proximal position. Preferably, portions 317a, 318a of springs 317, 318 maintain contact with cutting blade 314 regardless of the position of cutting blade 314. As such, distal end 314b is preferably of a length that contacts portions 317a, 318a at least when cutting blade 314 is in the intermediate and distal positions. In this way, electricity may be conducted from an energy source to cutting blade 314 then to first electrode 315 and second electrode 316 via springs 317, 318, as is described in more detail below.

Surgical device 300 includes an anvil 308 slidingly disposed in opening 306 between open and closed positions to capture tissue, such as a blood vessel, in opening 306. The vessel is preferably a side branch 6 of a vessel 5 to be harvested (see FIG. 9). In the open position, anvil 308 does not substantially interfere with the capturing of tissue in opening 306. While in the closed position, anvil 308 captures tissue between at least one clamping surface and at least a portion of slot edge 306a, preferably a distal portion 306b (FIG. 30C) of opening 306.

Referring to FIGS. 17 and 19, in a preferred embodiment, anvil 308 includes a first anvil 309 and a second anvil 310 formed of a plastic, such as polycarbonate. First and second anvil 309, 310 are elongated elements that are preferably of a length at least equal to the length of shaft 304, but could be of any length. First anvil 309 and second anvil 310 include anvil surfaces 309a and 310a located at the distal end of first and second anvils 309, 310 that serve to compress tissue captured within opening 306.

First anvil 309 and second anvil 310 form part of an anvil assembly 302 that also includes cutting blade 314, first electrode 311 and second electrode 312. End effector 301 includes anvil assembly 302 and shaft 304. Referring primarily to FIG. 19, first anvil 309 and second anvil 310 each are formed with recesses 309b, 310b that are configured to accept at least a portion of first and second electrodes 311, 312. During assembly, first electrode 311 is inserted into recess 309b to form one subassembly, and second electrode 312 is inserted into recess 310b to form a second subassembly. The attachment of the elements of assembly 302 may be by any method known in the art, but preferably, first electrode 311 and second electrode 312 are overmolded with first anvil 309 and second anvil 310, respectively. When the elements of anvil assembly 302 are assembled, they preferably leave a slot between first electrode 311 and second electrode 312 that permits cutting blade 314 to travel between the proximal, intermediate and distal positions therein. Preferably, anvils 309, 310, electrodes 311, 312 and cutting blade 314 are assembled by binding the elements together by a dielectric tube 315 that is shrink-wrapped around the assembly.

In an alternative embodiment, anvil 308 can comprise a second shaft within which first and second anvil 309, 310 are disposed. The second shaft can be slidingly disposed in first lumen 304a of first shaft 304. The second shaft is preferably a resilient medical grade material, such as stainless steel, and preferably a loose running fit is maintained between first shaft 304 and the second shaft. A spacer can be provided between first shaft 304 and second shaft 310, to define an annular space (not shown) between first shaft 304 and second shaft 310. The spacer is preferably a polymer that can act as a dielectric insulator. Further, rather than forming an anvil of separate pieces, anvil 308 may be formed of a single piece that is split at its distal end and is slotted to permit a cutting blade to slide therein.

When tissue is captured within opening 306 and clamped by anvil 308, radiofrequency energy may be supplied to the system so that the captured tissue can be ablated or desiccated. Because proximal portion 313b of tip 313 is recessed from distal end 304c of shaft 304, captured tissue is clamped at a location distal to opening 306 between anvil surfaces 309a, 310a and proximal portion 313b. The radiofrequency energy circuit for the clamp configuration is as follows: energy source to cutting blade 314 to electrodes 311, 312 to captured tissue to shaft 304 to the opposite pole of the energy source. Thus, when a blood vessel is captured within opening 306, the conduction path is through the blood vessel.

Once the tissue has been ablated or desiccated, cutting blade 314 can be advanced to the intermediate position to cut the tissue. Cutting blade 314 can be further advanced to the forward position, shown in FIG. 17, where cutting edge 314c protrudes from the distal portion 313a. This configuration permits the user to dissect tissue located distal of shaft 304 using cutting blade 314. In addition, because cutting blade 314 can act as an electrode in this configuration, surgical device 300 can be used for spot desiccation of tissue located beyond narrowed portion 313e of distal portion 313a. The radiofrequency energy circuit for the cut configuration is as follows: energy source to cutting blade 314 to tissue to shaft 304 to the opposite pole of the energy source. In this case, the conduction path is through tissue located outside opening 306 and shaft 304. Tabs 304f of shaft 304 may aid in providing a return circuit for RF energy supplied through cutting blade 314 and tissue distal to cutting blade 314.

The RF energy is preferably supplied from an electrosurgical generator (not shown), as is known in the art. The electrosurgical generator supplies the RF energy to the respective electrodes via wires 118a, 118b. The wires 118a, 118b are preferably routed through handle 110 within cable 119a and electrically coupled, such as by soldering or crimping, to the respective electrodes. In a preferred embodiment one of wires 118a, 118b is attached to proximal end 314a of cutting blade 314 and the other of the wires 118a, 118b is attached to proximal end 124a of second tube 124. A switch (not shown) is also preferably provided for energizing the electrodes with RF energy from the electrosurgical generator. The switch can be provided in handle 110 or in a foot switch or at some other location external to handle 110, as are known in the art.

Preferably, surfaces such as the exterior of tubes 123, 124 and shaft 304 are coated with a dielectric material to prevent a short between the electrodes of different polarity and also to prevent accidental cauterization of unintended tissue. Such coatings are well known in the art, and include polytetrafluorethylene (PTFE). It is important to note, that because the electrodes are offset from one another, thermal spread to unintended portions of the tissue or vessel being cauterized is minimized.

Anvil and Tip Shape

In the preferred embodiment, anvil 308 and cutting blade 314 can be retracted within shaft 304 to allow tissue to be placed into opening 306. Once the target tissue is in opening 306, anvil 308 can be advanced to clamp the tissue. As discussed above, when anvil 308 clamps tissue within opening 306, the distal end of clamp 308 mates with proximal portion 313b of tip 313. Referring to FIG. 17, proximal portion 313b of tip 313 includes mating surfaces 313g that are slightly rounded, one of which is depicted. Surfaces 309a, 310a of anvil 308 also have a slightly rounded shape that mate with the slightly rounded mating surfaces 313g of tip 313 when anvil 308 clamps tissue within shaft 304. This design permits tip 313 to provide a more uniform contact pressure distribution across the clamped tissue. Anvil and tip surface shapes were found by way of the following derivation.

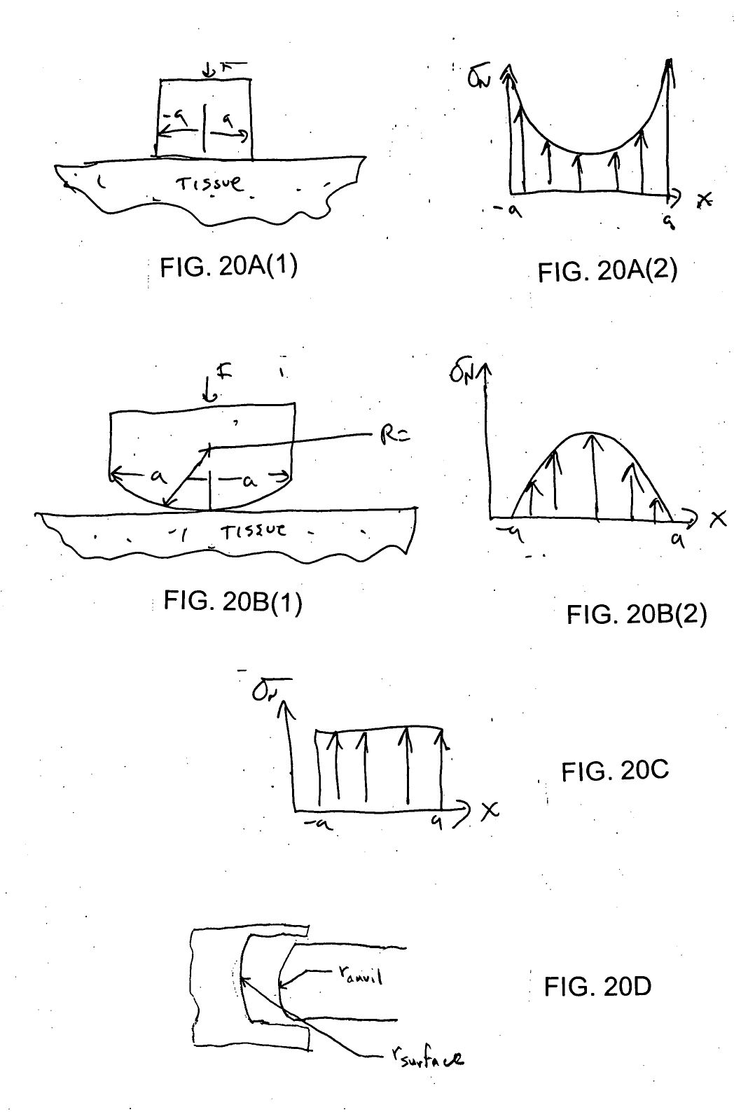

It is well known that a force applied by a flat bottom punch on a semi-infinite space, shown in FIG. 20a(1), produces a stress field with high concentrations at the edges, as shown in FIG. 20a(2). It is also well known that a force applied by round punch on a surface, shown in FIG. 20b(1), produces a parabolic (Hertzian) stress distribution, as shown in FIG. 20b(2). See Roark's Formulas for Stress and Strain (Warren Young 1989).

When sealing a side branch of a vessel, to produce good vessel sealing, a relatively uniform pressure distribution across the vessel is required to generate good coaption between the vessel walls. That is, a uniform pressure distribution causes opposing walls of the vessel to contact one another. As a result, when RF energy is applied to the vessel via electrodes 311, 312, the vessel seals more readily.

The ideal example of pressure distribution is shown in FIG. 20c. FIGS. 20a(2) and 20b(2) show that such a stress field can be created through a properly shaped tissue surface and indentor that combines stress distributions of FIGS. 20a(2) and 20b(2).

For an ideal embodiment, the ideal jaw surface takes the appearance of FIG. 20d. Hertz's analysis shows that the stress between two contacting bodies of arbitrary curvature is parabolic, and has a maximum given by:

$${σ}_{max} = \frac{1.5 F}{{π}^{a \hat{} 2}}$$

$$a = {0.721}^{3} √ {Fk}_{D} {C}_{ε}$$

$$where$$

$${C}_{ε} = \frac{1 - {r}_{1}^{2}}{{ε}_{1}} + \frac{1 - {r}_{1}^{2}}{{ε}_{2}}$$

where [symbol] ₁ and [symbol] ₂ are the elastic moduli of the materials. Roark's Formulas for Stress and Strain at 650. Where tissue is compressed by a plastic indentor as is the case with anvil 308, [symbol] ₂ is much greater than [symbol] ₁, as the modulus of elasticity of plastic ( [symbol] ₂) is approximately 500,000 psi, and the modulus of elasticity of tissue ( [symbol] ₁) is approximately 5,000 psi. As a result, the formula for constant C is simplified as follows:

$${C}_{ε} = \frac{1 - {r}_{1}^{2}}{{ε}_{1}}$$
which means that the ideal shape to produce a nearly flat stress distribution depends only on the tissue, and not the indentor.

A curvature mismatch, as shown in FIG. 20d, specifically where the radius of the mating surface (r_{surface})>radius of the anvil (rₐₙᵥᵢₗ) will produce a nearly flat pressure distribution. Various radii were tested to optimize the difference and it was found that favorable results were found when r_{surface} ranged between 1.05rₐₙᵥᵢₗ and 1.15rₐₙᵥᵢₗ, or a five to fifteen percent mismatch between the radii. In a preferred embodiment, the difference between r_{surface} and rₐₙᵥᵢₗ is approximately ten percent, giving a radius of the pocket or mating surface 313g of approximately 0.12″ and a radius of the anvil is approximately 0.11″. This difference has been shown empirically to produce more effective sealing of vessels.

Actuation

Referring now to FIGS. 13 and 14, surgical device 300 includes a control mechanism 320 that actuates each of the multitool functions. As such, control mechanism 320 (a) moves shaft 304 between the proximal and distal positions, (b) moves anvil 308 between the open and closed positions, (c) moves cutting blade 314 between the proximal, intermediate and forward positions. Control mechanism 320 is particularly advantageous in that it simplifies the actions the user needs to make to operate surgical tool 300. While the preferred embodiment provides a single actuator for actuating each of the different functions of surgical device 300, one skilled in the art will understand that surgical device 300 could have two or more actuators to perform an action performed by control mechanism 320. Control mechanism 320 preferably provides high levels of force to anvil 308 when actuated using low levels of force when the mechanism moves in one direction, and provides large displacements at low forces when the mechanism moves in the opposite direction.

Preferably, control mechanism 320 includes a button 115 that is movably disposed in handle 110, and operatively connected to shaft 304, anvil 308, and cutting blade 314. Moving button 115 a first predetermined amount moves shaft 304 between the proximal and distal positions; moving button 115 a second predetermined amount moves anvil 308 between the open and closed positions; and moving button 115 a third predetermined amount further moves cutting blade 314 between the open and closed positions.

Referring to FIGS. 13 and 14, preferably button 115 is attached to a yoke 321 that extends through slot 116 of handle 110. Yoke 321 preferably includes a rod 327 that extends longitudinally, and a stem 322 that extends upwardly from the proximal end of rod 327. Stem 322 is configured to extend through slot 116 of handle 110 and matingly engage with button 115, preferably by a friction fit, but alternatively by any means known to one skilled in the art. Yoke 321 is attached to a compressor 330 (described below), preferably at a distal end of rod 327. Any attachment mechanism known in the art may suffice, but a preferred embodiment includes a first projection 323 that projects from the distal end of rod 327, and a second projection 324 that projects form rod 327 at a position offset from first projection 323. An intermediate portion 326 extends longitudinally between first projection 323 and second projection 324. A tab 325 extends laterally from rod 327, most preferably from second projection 324.

Preferably, control mechanism 320 includes a sled 350, a flexure mechanism 340, and a compressor 330. Sled 350 is sized and configured to be disposed within compartment 111 of handle 110 and is slidable between a proximal position to a distal position within compartment 111. Flexure mechanism 340 is disposed and movable within sled 350 and is compressed by compressor 330, which is disposed in part about flexure mechanism 340 to compress flexure mechanism 340 from a first, relaxed configuration to a second, straightened configuration. A yoke 321 serves to translate movement from the button 115, to which it is attached on one end, to compressor 330, to which it is attached on another end. Each of yoke 321, compressor 330, flexure mechanism 340 and sled 350 may be made from a suitable plastic known to those skilled in the art, such as a polycarbonate.

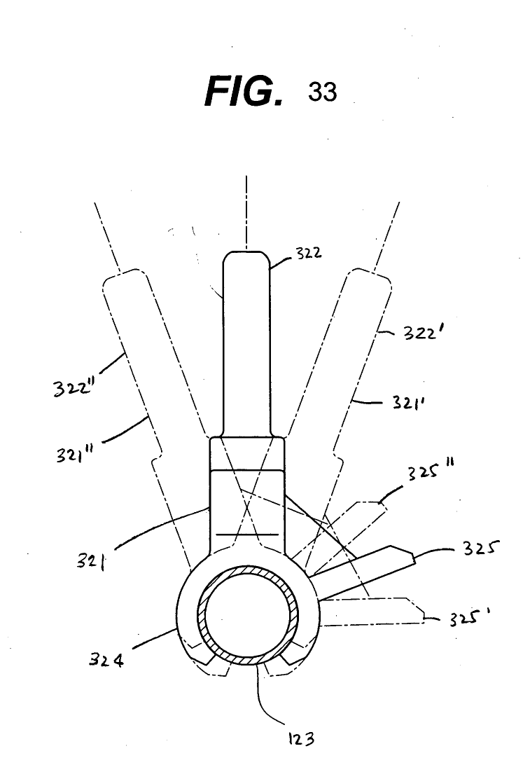

Referring to FIG. 33, a rear plan view of yoke 321 is depicted in three different positions. In the position shown in dark lines, yoke 321 is positioned in third track 117c, which is substantially aligned with medial plane M. Yoke 321′ is rotated clockwise about tube 123 when stem 322 is disposed within first track 117a. In this clockwise position, tab 325′ compresses sled lock 360, thereby permitting sled 350 to move within compartment 111 of handle 101. Yoke 321″ is rotated counter-clockwise about tube 123 when stem 322 is disposed within second track 117b. When stem 322 is disposed within second track 117b and third track 117c, tab 325′ (325) is no longer disposed above sled lock 360, which thereby is permitted to move into notch 110g within handle 101, preventing movement of sled 350 with respect to handle 110.

Referring to FIGS. 13, 14 and 21, sled 350 is disposed within compartment 111 formed in proximal end 110d of handle 110. Sled 350 has an opening 351 at a distal end 350a to accommodate the proximal end 304b of shaft 304, which is preferably attached to distal end 350a at that location. Sled 350 includes guides 352a and 352b laterally offset from one another that cooperate with projections 112a and 112b that extend upwardly from bottom surface 112 of handle 110. Guides 352a and 352b of sled 350 ride upon projections 112a and 112b of handle 110 when sled 350 moves between a proximal position and a distal position within compartment 111. Sled 350 also includes a distal semicircular support 353a and a proximal semicircular support 353b for supporting tube 123, which is fixed to proximal end 110c of handle 110. Tube 123 provides a lumen for passing an endoscope through and also serves as a rail upon which sled 350 and compressor 330 travel. Sled 350 is thus constrained between tube 123 and projections 112a and 112b of handle 110 as sled 350 moves between a proximal position and a distal position within compartment 111.

Sled 350 also has one or more openings that communicate with the area between projections 112a and 112b beneath sled 350 to accommodate wiring that connects an energy source to the electrodes. For example, sled 350 has a proximal opening 354 for permitting wire 118 to be attached to proximal end 314a of cutting blade 314.

Sled 350 also includes at least one feature that cooperates with compressor 330 when compressor 330 is moved from a proximal position to an intermediate position. Preferably sled 350 includes a detent 355 formed in a side wall 350c of sled 350 that includes a projection 355a to mate with a recess in compressor 330 when compressor 330 is in the intermediate position. An inner wall 356 extends from bottom wall 350b and back wall 350d of sled 350. Inner wall 356 includes a top surface 356a, and a cam 356b that extends upwardly from top surface 356a. Inner wall 356 includes an opening 356c configured to accept a tab 325 of yoke 321 when compressor 330 is in the intermediate position.

Sled 350 preferably includes a sled lock 360 that is configured to be disposed within a sled lock chamber 358 formed by inner wall 356 and members 357a and 357b that extend from a side wall 350e to inner wall 356. Sled lock 360 includes a spring 361 that is at least partially disposed about a stake 359 that extends upwardly from bottom wall 350b within sled lock chamber 358, and a button 362 having an orifice that houses a portion of spring 361. Button 362 preferably has ears 362a, 362b that ride in slots within members 357a, 357b to maintain button 362 in a centered position within sled lock chamber 358.

Referring to FIG. 14, a perspective view of handle 110 with handle half 110a rotated to more clearly depict underside 110e of handle half 110a. A ramp 110h extends from underside 110e and tapers from a first height 110i to a second, shorter height 110j. Ramp 110h has a notch 110g at a location corresponding to the location of tab 325 of yoke 321 when sled 350 is at the distal position.

Tab 325 is configured so as to be disposed at least partially over button 362 of sled lock 360 and within opening 356 (FIG. 19) when sled 350 is in the proximal or IN position so as to compress button 362 against spring 361. Thus, sled lock 360 is held in a compressed state by tab 325 when sled 350 is in the IN position as yoke 321 is permitted only to move along first track 117a. Sled lock 360 is permitted to assume an uncompressed state only when sled 350 is in the distal or OUT position.

Control mechanism 320 also includes compressor 330 that is at least partially disposed about flexure mechanism 340. Referring to FIGS. 21-23, flexure mechanism 340 includes a distal end 340a that is attached to anvil assembly 302 of surgical device 300 so that as distal end 340a of flexure mechanism 340 moves distally or proximally, anvil assembly 302 follows. Flexure mechanism 340 includes a proximal end 340b that has a first post 341 and a second post 342 that extend proximally therefrom. Posts 341, 342 are configured to accept, or in the alternative are attached to, springs 343, 344, respectively. Springs 343, 344 may be coil springs or flat springs or any other type of spring known to those skilled in the art. Spring 343 is contained between post 341 at a distal end of spring 344 and a post 350g that projects from back wall 350d on one side of opening 354. Similarly, spring 344 is contained between post 342 at a distal end of spring 344 and a post 350h that projects from back wall 350d on the other side of opening 354. Flexure mechanism 340 and springs 343, 344 are constrained within sled 350.

The spring constant of springs 343, 344 are preferably chosen such that a sufficient clamping force must be reached before cutting blade 314 is advanced. This ensures a proper ligation of a vessel captured in opening 306 before transection by the cutting edge 314c of cutting blade 314.

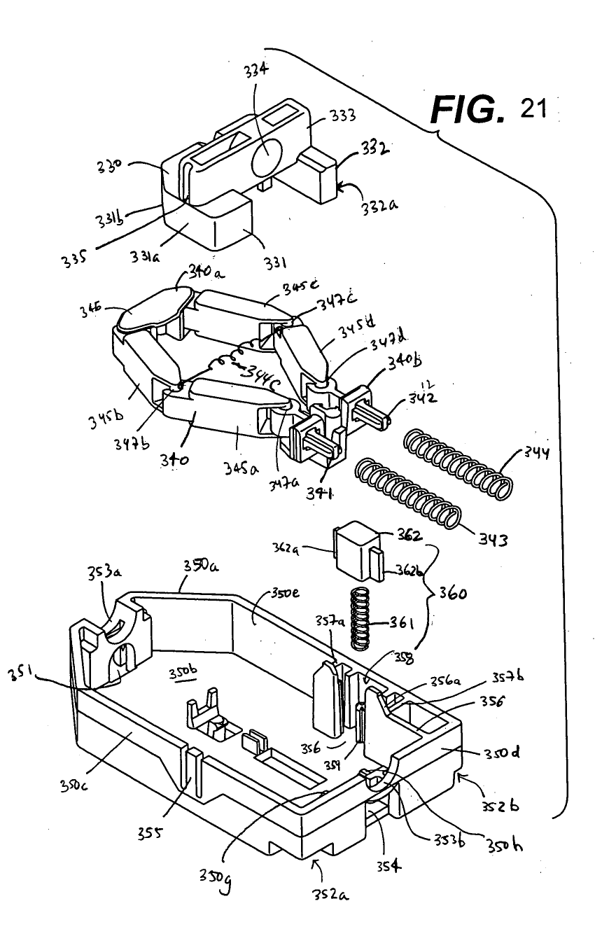

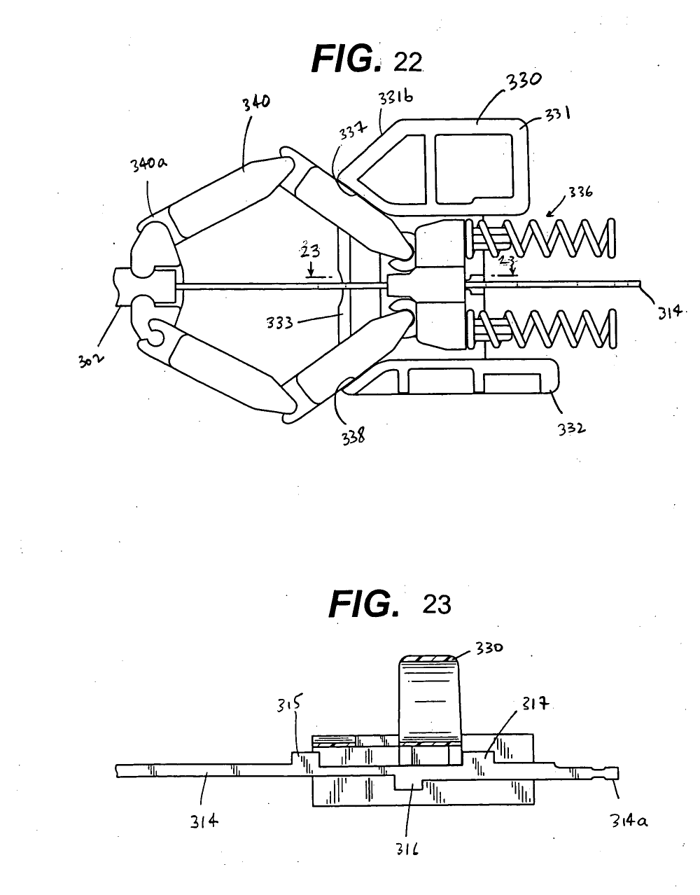

Referring to FIGS. 21 and 22, compressor 330 preferably includes a first leg 331 and a second leg 332 spaced apart from first leg 331. First leg 331 and second leg 332 are connected by a cross member 333 that is preferably substantially perpendicular to first and second legs 331, 332. Preferably, cross member 333 of compressor 330 is captured between first and second projections 323, 324 of yoke 321. In a preferred embodiment, first and second projections 323, 324 each take the form of a semi-cylinder sized to snap-fit onto first tube 123 at a location on either side of cross member 333. As such, first and second projections 323, 324 ride on first tube 123 when yoke 321 is moved between a proximal position and a distal position. In addition, first and second projections 323, 324, and as a result yoke 321, are rotatable through with respect to first tube 123. The rotation of yoke 321 is constrained by cross member 333, which is configured to contact the underside of intermediate portion 326 of yoke 321 when yoke is rotated a desired amount.

Together with first and second legs 331, 332, cross member 333 and bottom surface 350b of sled 350 form a channel 336 for compressing flexure mechanism 340 between an expanded configuration, a flexed configuration, and a straightened configuration. First and second legs 331, 332 have distal surfaces 337, 338, respectively that are configured to direct flexure mechanism 340 into channel 336. Preferably, distal surfaces 337, 338 are angled such that the proximal end of flexure mechanism 340 smoothly enters channel 336.

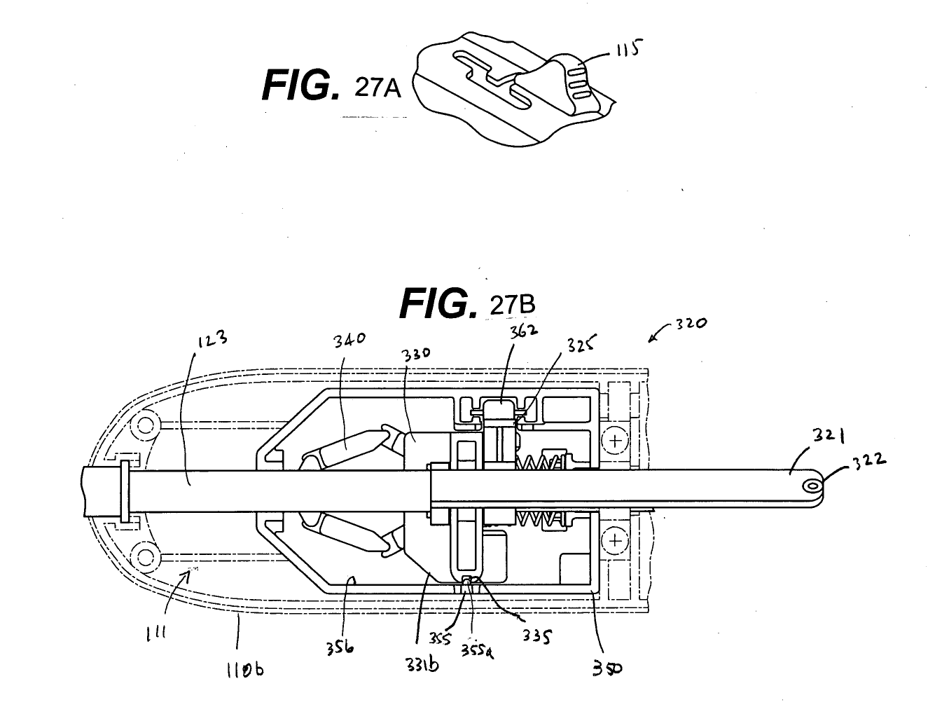

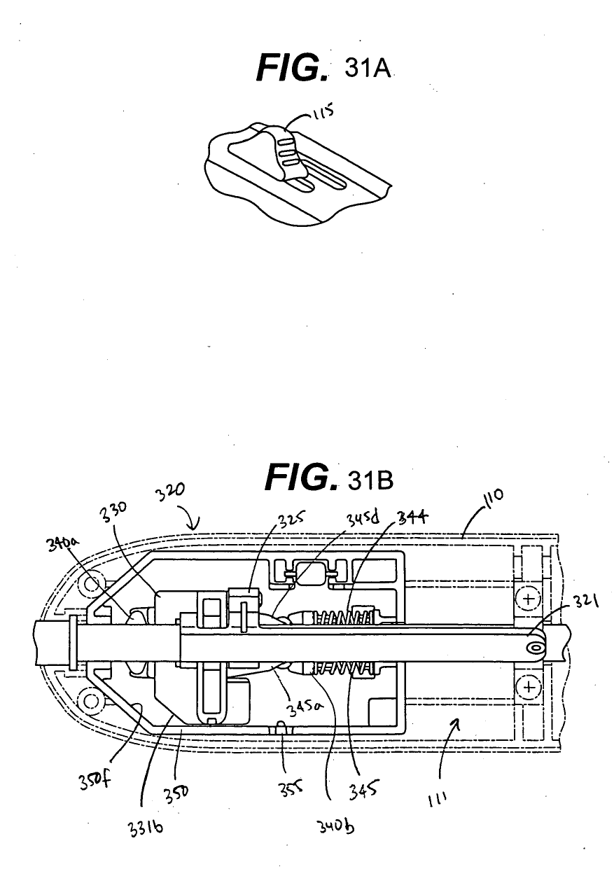

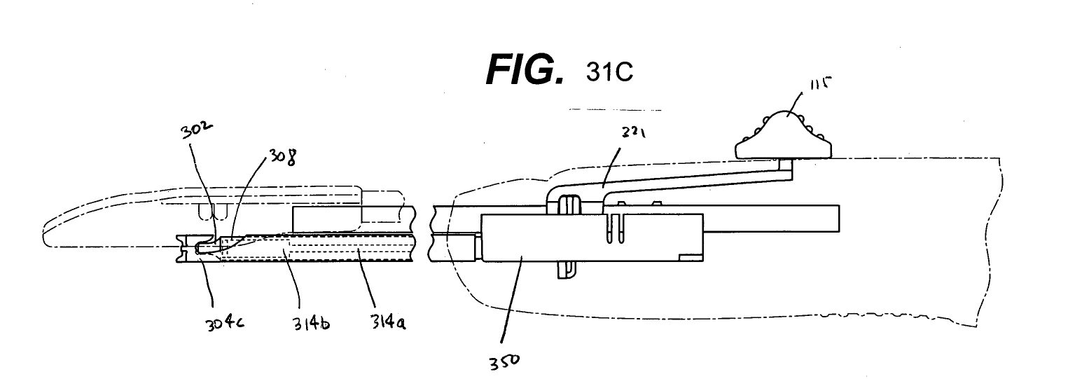

Cross member 333 includes a bore 334 sized to permit tube 123 to pass therethrough. Referring to FIG. 27, cross member 333 also includes a recess 335 for cooperating with projection 355a of detent 355 when compressor 330 is in the intermediate position. First and second legs 331, 332 are spaced such that outer wall 331a of first leg 331 and outer wall 332a of second leg 332 slidingly ride within inner wall 356 and side wall 350c of sled 350. First leg 331 includes a mating surface 331b shaped to mate with an inner wall portion 350f of sled 350 when compressor 330 is in the distal/forward position (FIG. 31B).

In a preferred embodiment, and referring to FIG. 21, flexure mechanism 340 is a four-bar linkage that can be made to lengthen or shorten by passing flexure mechanism 340 through channel 336 of compressor 330. Flexure mechanism 340 includes a first rod 345a, pivotably attached on one end at pivot 347a to proximal end 340b, and at the other end at pivot 347b to a second rod 345b. Second rod is pivotably attached to cross member 346, which in turn is pivotably attached to an end of a third rod 345c. Third rod 345c is pivotably attached on another end at pivot 347c to a fourth rod 345d, which in turn is pivotably attached at pivot 347d to proximal end 340b. Flexure mechanism 340 moves between a proximal position, an intermediate position and a distal position. Pivots 347a-347d are preferably lubricated to permit rods 345a-345d to easily pivot about pivots 347a-347d. In an alternative embodiment, pivots 347a-d can be living hinges.

Flexure mechanism 340 could be built as a linkage with four rigid bars, connected by pin joints and therefore would have a stiffness (rotational friction) very close to zero. The flexure can also be made as a one-piece element with living hinges at its pivot points. It is also possible to introduce arbitrary force displacement profiles at the jaw and button by varying the spring rate and preload of the springs. In a preferred embodiment, where first and second anvil 309, 310 and first and second electrodes 311, 312 have an area of approximately 0.00714 in², flexure mechanism 340 and springs 344, 345 are adjusted to produce a clamping force of between 2 to 3 lbs., which generates a pressure range of between 280 and 420 psi at anvil assembly 302, with a maximum button force F_{B} of less than 2 lbs., and preferably about 1.5 lbs.

One method of modifying the stiffness of flexure mechanism 340 is to introduce a spring 344c that spans from rod 345a to 345d. Varying the stiffness and/or preload of spring 344c will vary the force displacement curve of button 115 in this direction.

A further feature of flexure mechanism 340 is that it can be used as a locking mechanism as well because it is an “over-center” mechanism. If rods 345a-345d are pushed slightly past the straight position by sizing channel 336 of compressor 330 to produce such an effect, they will lock and cannot be opened using a control rod, in this case cutting blade 314. Conversely, preventing flexure mechanism 340 from reaching this state will ensure that it can always be opened using the control rod (cutting blade 314).

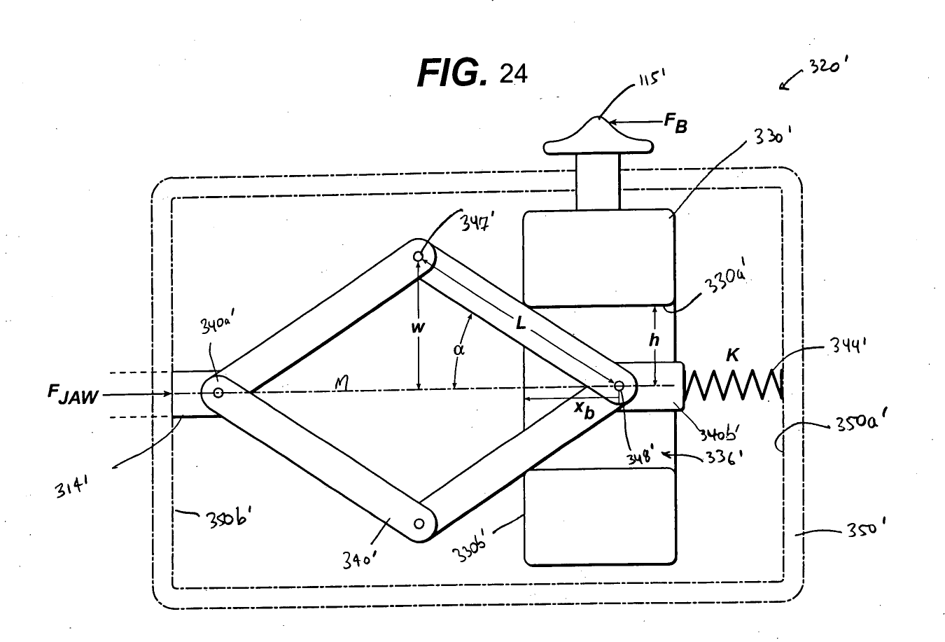

FIG. 24 shows a sketch of a control mechanism 320′, idealizing it as a four-link mechanism with rods of equal lengths. A structure 350′ having a first surface 350a′ and a second surface 350b′ houses the control mechanism. The control mechanism includes four-link mechanism 340′, a compression member 330′ having a channel 336′ for compressing four-link mechanism 340′, and a spring 344′ constrained at one end by surface 350a′ and at the other end by proximal portion 340b′. A distal end 340a′ of four-link mechanism 340′ is constrained by surface 350b′ of structure 350′ and drives a control rod 314′, which is slidable within surface 350b′ of structure 350′. Alternatively, mechanism 340′ is constrained by the limited travel of control rod 314′; i.e., control rod 314′may be limited in its travel by a stop located inside or outside structure 350′.

One can predict the required actuation forces, F_{B}, and anvil or jaw force, F_{JAW}, from the following equations:
F_{JAW}=KL(cos α−cos αₒ)
αₒ=asin(W/L)
F_{B}=KL²/l[(cos α−cos αₒ)−sin ²α
α=atan(h/x_{b})
l=(h²+x_{b}²)^{1/2}
where K is the spring constant of spring 344′, L is the length of a rod of four-link mechanism 340′, α is the angle between a medial axis M and a pivot 347′ of four-link mechanism 340′, W is the distance between medial axis M and pivot 347, h is the distance between medial axis M and the inner surface 330a′ of channel 336′, and x_{b} is the distance between proximal pivot point 348′ and the distal surface 330b′ of compressor 330′. Note that, while rods have been assumed to be of equal lengths, the calculation can readily be generalized to the case where the links have unequal lengths.

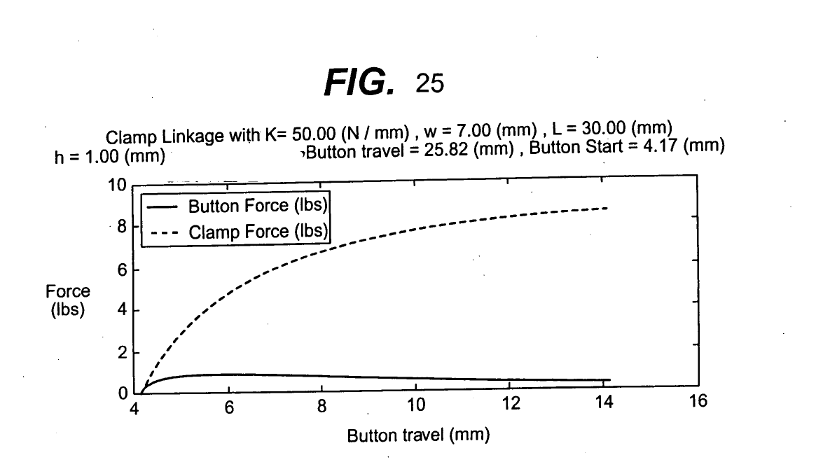

Examination of equation 1 shows that as the linkage gets flatter, the force amplification increases dramatically, making it possible to produce very large output forces with very small input forces. FIG. 25 shows the results of a computer simulation for one design of the mechanism, where the values of the variables are shown on the charts, showing that a nearly 10:1 input force to output force ratio has been achieved.

Referring to FIG. 24, control mechanism 320′ preferably provides high levels of force to an anvil located at the end of control rod 314′, while requiring only low levels of force at actuator button 115′ when button 115′ is moved in the direction indicate by arrow F_{B}. When button 115′ is moved in the direction opposing arrow F_{B}, control system 320′ provides large displacements at low forces. FIG. 25 demonstrates that the peak button force F_{B} occurs early in the travel of the mechanism and is less than one-eighth of the jaw force. It also demonstrates that button force F_{B} remains low, and relatively constant, throughout the travel of button 115′ because of the varying motion ratios.

Possible applications of this control mechanism include clamping and control mechanisms for bipolar surgical instruments, stapling instruments and clamping instruments. In addition, the mechanism could also be readily used to tension a cable that is used to lock a segmented heart stabilizer arm in place with a minimum of input force. The mechanism provides the ability to produce large forces with low actuation forces in one direction with large displacements and low forces in the other direction.

Further, the stiffness of control mechanism 320 is variable in both directions. In the direction opposing arrow F_{B} in FIG. 24, the apparent stiffness of button 115 is governed by the stiffness of spring 344′. In the direction of arrow F_{B}, the stiffness of button 115 is governed by the stiffness of flexure mechanism 340′. By varying the preload and stiffness of spring 344′ and flexure mechanism 340′, it is possible to generate arbitrary displacement profiles.

Conversely, sliding compressor 330′ proximally in the direction opposing arrow F_{B}, compressor 330′ comes into contact with the control rod 314′ which in turn pulls an end effector proximal; e.g., a jaw open or a cutting blade proximally. The jaw continues to open (or the blade continues to travel proximally) until flexure mechanism 340′ expands or flattens to reach the state shown in FIG. 30B, for example. Thus, one sees that as compressor 330′ slides in the direction of arrow F_{B}, the stiffness of control mechanism 320′ is set by spring 344′ and the force ratio is governed by the motions of flexure mechanism 340′, while as compressor 330′ slides in the direction opposing arrow F_{B}, compressor 330′ pulls directly on control rod 340b′ and the stiffness of the mechanism is governed by the stiffness of the joints of flexure mechanism 340′.

Method of Actuation

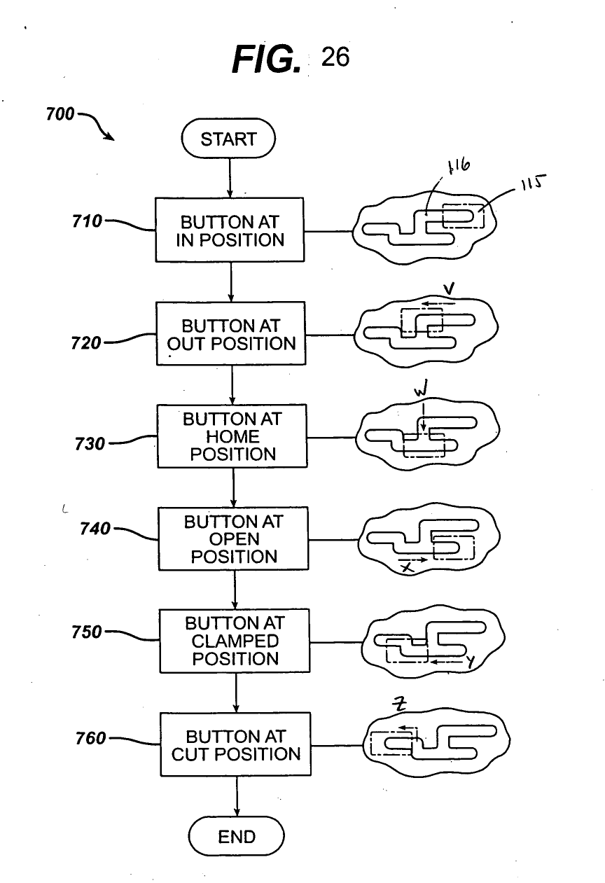

Referring to FIG. 26, a schematic depicts the different positions of button 115 within slot 116. In a typical operation 700, the user moves button 115 from an IN position 710 in a direction V to an OUT position 720 which moves shaft 304 to OUT position 720. Button 115 is then permitted to move in a direction W to a HOME position 730, which permits the user to move button 115 in a direction X to an OPEN position 740. At OPEN position 740, the user can maneuver the surgical device such that tissue is disposed within opening 706 of shaft 304. At this stage, the user can move button 115 to a CLAMPED (or closed) position 750 where anvil 308 clamps tissue within opening 306. Finally, the user can move button 115 to a CUT position where cutting blade 314 cuts tissue clamped in opening 306 and extends distally from tip 313 of surgical tool 300. At this point, cutting blade 314 and anvil 308 can be retracted by moving button 115 to OPEN position 740, and surgical device 300 is ready for another use.

FIGS. 27-32 describe each of the positions outlined in FIG. 26 in more detail. FIGS. 27A-27C depict, respectively, the positions of button 115, control mechanism 320 and end effector 301 when multitool 100 is in the IN position 710. The user generally starts using multitool 100 with button 115 at the most proximal position within slot 116 at the IN position 710. At this stage, as depicted in FIG. 27B, sled 350 is in the most proximal position within chamber 111 of handle 101. Within sled 350, compressor 330 is in its intermediate position: projection 355a of detent 355 is seated within recess 335 of compressor 330; flexure mechanism 340 is in its flexed position; and tab 325 of yoke 321 is disposed at least partially between button 362 of sled lock 360 and underside 110e. Yoke 321 is rotated slightly clockwise relative to the medial plane M as stem 322 is positioned within first track 117a.

Referring to FIG. 27C, end effector 301 is depicted in the IN position. Shaft 304 is in the proximal position disposed beneath head 53 of retractor 50 proximal to first paddle 62 of first manipulator 60. Anvil 308 is in its closed position obstructing opening 306.

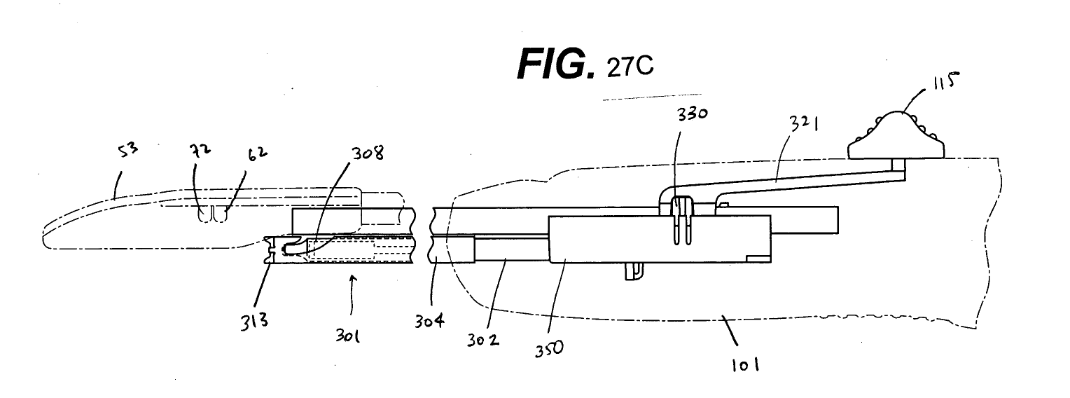

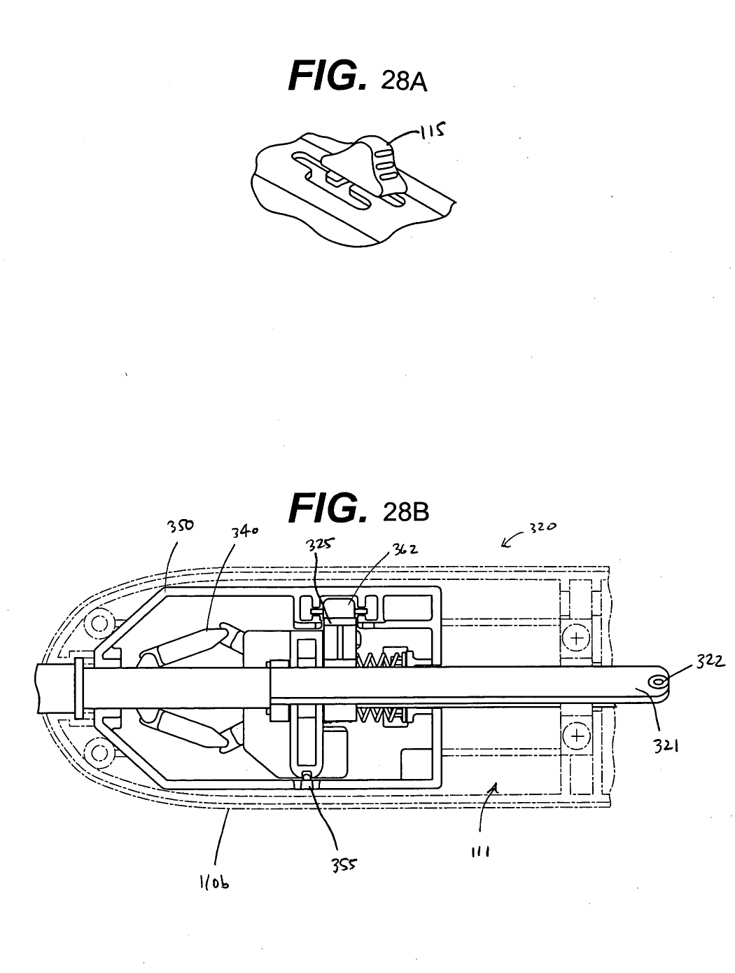

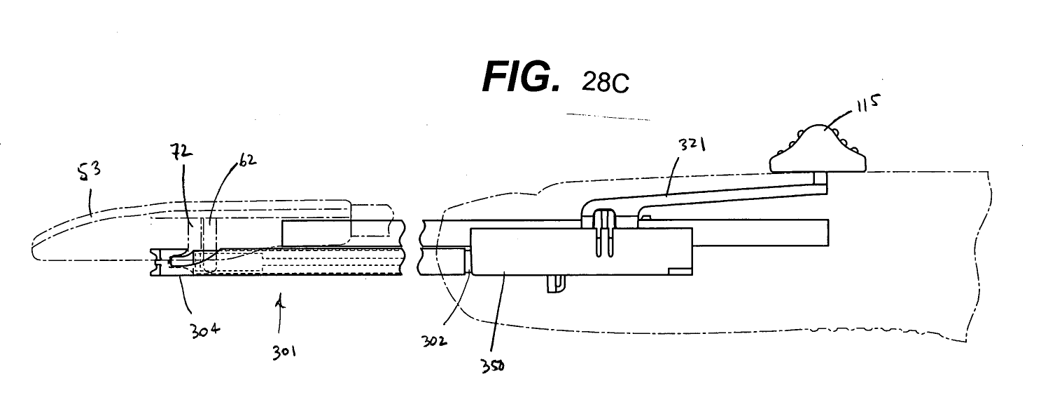

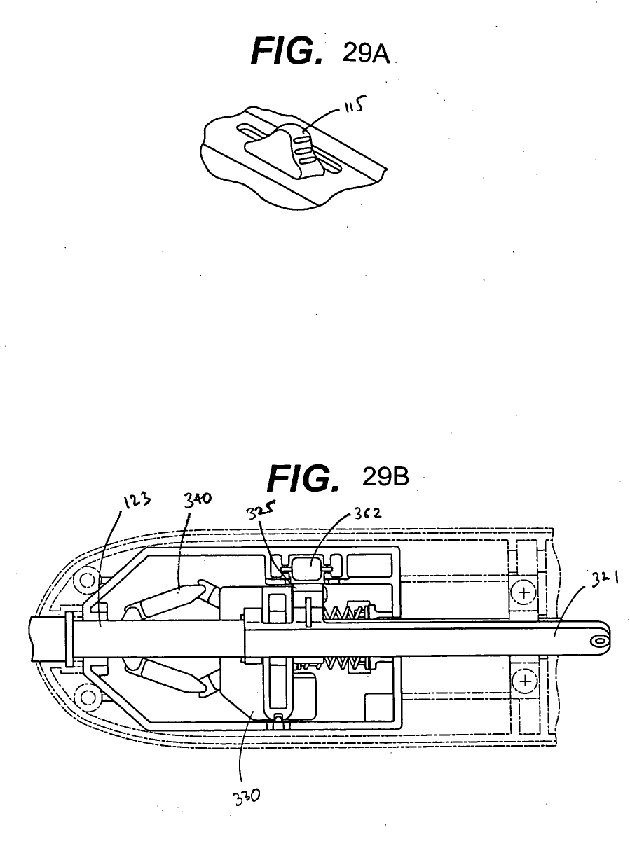

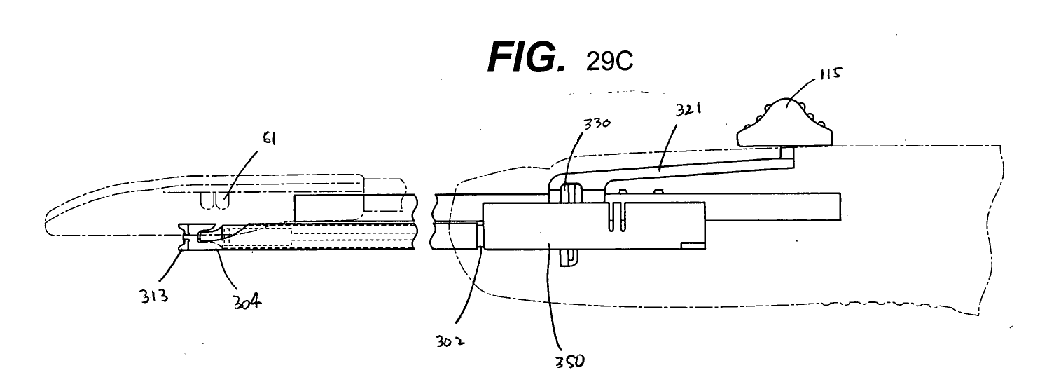

FIGS. 28A-28C depict, respectively, the positions of button 115, control mechanism 320 and end effector 301 when multitool 100 is in the OUT position 720. As the user moves button 115 from the IN position 710 to the out position 720 by moving button 115 distally within first track 117a, tab 325 of yoke 321 is captured within opening 356 of sled 350. As such, the movement of button 115 is translated to yoke 321 directly to sled 350, and sled 350 is moved from the proximal position to the distal position. Because proximal end 304b of shaft 304 is connected to distal end 350a of sled 350, as sled 50 moves distally, shaft 304 slides distally within tube 124, such that opening 306 is disposed beneath paddles 62, 72 of retractor 50 (FIG. 29C) or to either side of paddles 62, 72 (FIG. 28C), if paddles 62, 72 are positioned in their extended position, or to one side of either paddle 62, 72, if one of paddles 62, 72 are positioned in their extended position. Anvil 308 remains in its closed position obstructing opening 306.

As button 115 is moved distally from the IN position 710 to the OUT position 720 within first track 117a, tab 325 gradually moves up ramp 110h of handle half 110a (FIG. 14) until tab 325 reaches notch 10g. At this point, button 115 is in the HOME position 730 depicted in FIGS. 29A-29C. At this position, fourth track 117d permits button 115 and yoke 321 to move laterally in a direction W (FIG. 26), and as a result, yoke 321 rotates along with tab 325 in a counterclockwise direction about tube 123 to a position where tab 325 no longer contacts (or compresses) sled lock 360. As such, sled lock button 362, under the force of spring 361, enters notch 110g to lock sled 350 in the distal OUT position and prevent sled 350 from moving proximally. As a result, shaft 304 is also locked in the out position. In addition, when tab 325 rotates counterclockwise, tab 325 is freed from the constraint of opening 356 of sled 350, thereby permitting movement of compressor 330 within sled 350. As with the IN and OUT positions 710, 720, anvil 308 remains in its closed position obstructing opening 306.

Next, the user can move button 115 to the OPEN position 740 depicted in FIGS. 30A-30C. In traveling from the HOME position 730 to the OPEN position 740, the user moves button 115 proximally within second track 117b, and yoke 321 (shown in its counterclockwise-tilted position) is no longer constrained by tab 325 to sled 350, directly acts on compressor 330 to dislodge detent 355 from recess 335 of compressor 330. In so doing, compressor 330 moves proximally within sled 350, thereby disengaging compressor 330 from flexure mechanism 340. As is depicted in FIG. 23, as compressor 330 moves proximally, the compressor engages flag 317 of cutting blade 314 thereby pulling cutting blade 314 proximally. The proximal movement of cutting blade 314 in turn pulls anvil assembly 302 proximally, which has the effect of both opening flexure mechanism 340 moves from its flexed position (FIG. 29B) to its expanded position (whereat rods 345a and 345d contact side walls 350c and 350e of sled 350, respectively), and moving anvil assembly 302 to the OPEN position. Referring to FIG. 30C, anvil assembly 302 is shown substantially disposed within shaft 304, thereby exposing opening 306.

Referring to FIGS. 31A-31C, button 115, control mechanism 320 and end effector 302 are shown, respectively, in the CLAMPED position 750. Moving button 115 in a distal direction Y (FIG. 26) from the OPEN position 740 to the CLAMPED position 750 within second track 117b moves yoke 321 distally. Yoke 321 acts directly on compressor 330 and moves compressor 330, first to a position like that depicted in FIG. 27B, where flexure mechanism 340 is in the flexed configuration, and detent 355 is captured in recess 335, and then to a more distal position where flexure mechanism 340 is in the straightened configuration. As compressor 330 moves distally, it engages flexure mechanism 340 and begins to “squeeze” flexure mechanism 340 flat. As flexure mechanism 340 passes through channel 336, the rods 345a, 345b, 345c and 345d of flexure mechanism 340 are pressed inward at pivots 347b and 347c, causing the overall length of flexure mechanism 340 to increase. That is, as flexure mechanism 340 flattens, it effectively gets longer. Flexure mechanism 340 moves anvil assembly 302 distally until anvil surface 309a, 310a contacts proximal portion 313b of tip 313. Once contact is made, the force generated by the contact distal end 340a of flexure mechanism 340 is greater than the spring force provided by springs 344, 345. As a result, when compressor 330 is moved further distally, flexure mechanism 340 continues to flattened, but instead of distal end 340a moving distally, proximal end 340b of flexure mechanism 340 moves proximally and engages springs 344, 345, which generates a reactive spring force. Any further compression of flexure mechanism 340 by compressor 330 causes flexure mechanism 340 to again increase in length and thereby compress springs 343, 344 until flexure mechanism 340 reaches the fully compressed state as shown in FIG. 31C. The reactive spring force provides the clamping force for surgical device 300, thereby clamping tissue disposed within opening 306 against proximal portion 313b of tip 313 and distal portion 306b of opening 306. Cutting blade 314 remains in its proximal position as compressor 330 travels between flag 317 and flag 315 (FIG. 23) and does not interact with cutting blade 314 when moving from the OPEN to the CLAMPED position.

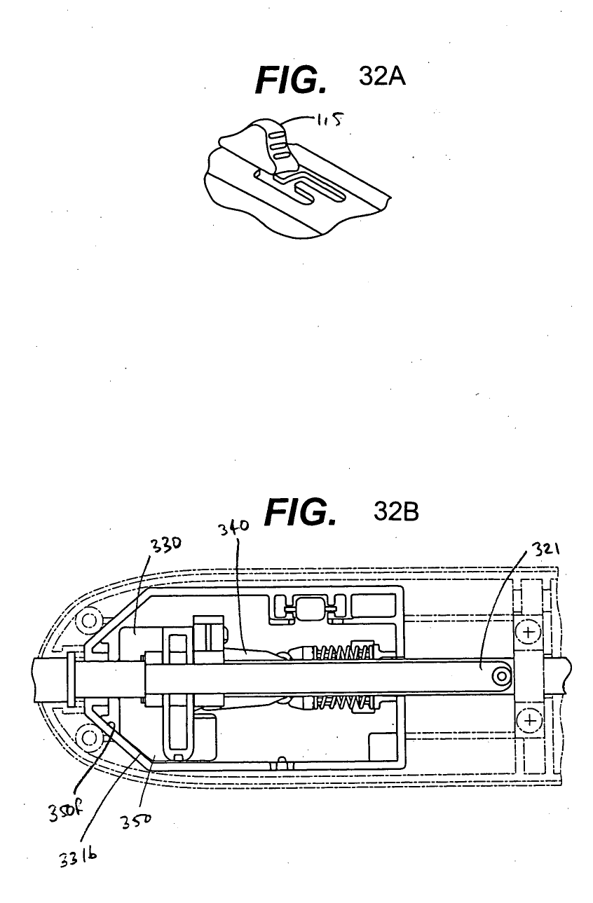

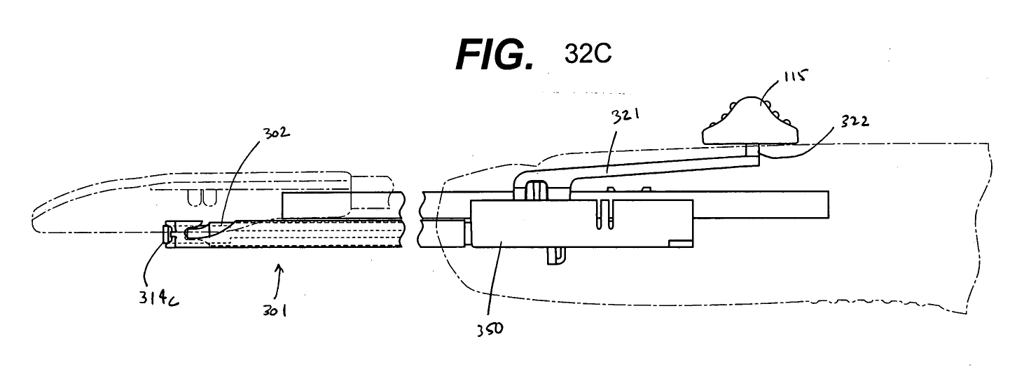

Referring to FIGS. 32A-32C, button 115, control mechanism 320 and end effector 302 are shown, respectively, in the CUT position 760. As the user moves button 115 from the CLAMP position 750 within second track 117b to the CUT position 760 within third track 117c, button 115 is moved distally in a direction Z (FIG. 26). At this stage, sled 350 and flexure mechanism 340 are at their distal positions. Moving button 115 distally directly acts on yoke 321, which in turn acts on compressor 330. As compressor 330 moves distally, it engages flag 315 of cutting blade 314 (FIG. 23) and moves cutting blade 314 distally until cutting edge 314c of cutting blade 314 travels through proximal portion 313b of tip 313 to cut the desiccated tissue. If the user maintains pressure in the CUT position, leading edge 314c of cutting blade 314 remains exposed beyond distal portion 313a of tip 313, thereby permitting the user to use cutting edge 314c for sharp dissection and/or spot coagulation.

Method of Use

To utilize system 600, a physician or physician's assistant determines the location of a vessel to be dissected, and makes an incision in the patient. The user then inserts retractor 50 or a separate dissection device into the incision and bluntly dissects the tissue surrounding the vessel using working head 53. If the intention is to extract vessel 5 (see FIG. 9), it is preferable to dissect as much tissue from around the vessel as possible. The user manipulates retractor 50 to advance working head 53 along vessel 5, separating tissue from vessel 5 and providing a working space for accessing and visualizing vessel 5 and a plurality of side branches, one of which is shown in FIG. 9 as reference numeral 6.

The user then uses multitool instrument 100 to free vessel 5 from the surrounding tissue and isolate side branches of the vein that must be ligated prior to removal of vessel 5 from the patient's leg. As noted above, multitool instrument may be located above vessel 5 and below shaft 52 of retractor 50, when docked with retractor 50, or may be positioned below shaft 52 of retractor 50 in an undocked configuration.

Referring to FIG. 9, the user manipulates either paddle 62 and/or 72 of retractor 50 to position vessel 5 away from multitool 100 permitting the user to dissect, clamp, coagulate, and cut tissue within working space 57. In particular, when side branches 6 are encountered, the user can manipulate vessel 5 using, for example paddle 62 of retractor 50 such that vessel 5 is protected. In this manner, side branches 6 are isolated and exposed and surgical device 300 introduced via multitool 100 (or through cannula 252) can cauterize and cut side branch 6 without damaging vessel 5.

During the dissection of vessel 5, whenever a side branch 6 is encountered, vessel 5 can be manipulated to protect it by retractor paddles 62, 72. Whether multitool is in the docked or undocked configuration, button 115 is moved from the IN position 710 to the OUT position 720 to move shaft 304 to its forward position. When in the docked configuration, the distal end of shaft 304 is disposed beneath paddles 62, 72 when it is in its forward position. Button 115 is then moved to the OPEN position 740 to retract anvil assembly 302 within shaft 304 to a position that exposes opening 306 of shaft 304.

At this point, shaft 304 of multitool 100 is positioned such that side branch 6 is captured within opening 306. Button 115 is then moved to the CLAMPED position 750, which causes anvil assembly 302 to move distally within shaft 304 to clamp side branch 6 in opening 306. Preferably, side branch 6 is clamped between clamping surface 308a and an edge of distal portion 306b defining opening 306. Once side branch 6 is captured and clamped, RF energy is preferably applied to the first electrode 311 and second electrode 312 by activating a switch (typically a foot switch) to cauterize side branch 6. Cauterization of side branch 6 sufficiently ligates side branch 6 such that it can be safely severed.

Side branch 6 is then severed by moving button 115 from the CLAMPED position 750 to the CUT position 760, thereby moving cutting edge 314c of cutting blade 314 distally through opening 306 and at least partially into slot 313c to sever cauterized side branch 6. Button 115 can then be moved back to the OPEN position 740 to be ready to perform ligation and transection of the next side branch.

The harvesting procedure continues in this manner until vessel 5 is hemostatically isolated from the surrounding tissues and blood supply along the portion to be harvested. Once the user completes the dissection and vessel 5 is freed of its surrounding tissue, retractor 50 can be withdrawn through the incision. Vessel 5 can then be removed from its native location and prepared for use in a coronary bypass procedure, for example.

It should be understood that paddles 62, 72 can operate in tandem or can be manipulated such that they work independently of one another. For example, paddle 62 can be extended independently of paddle 72 as it is positioned distally to paddle 72. Paddle 72 may also bypass paddle 62 by first extending each paddle to a position forward of the distal end of cannula 52, rotating paddle 72 such that it does not interfere with paddle 62, and then retracting paddle 62 into the stowed position within cannula 52.

While system 600 is especially suited for vessel harvesting for a coronary artery bypass procedure (a description of which is found in U.S. Pat. No. 6,616,661, and is hereby incorporate by reference), it is not limited to this surgical procedure. Of course, while described as being used together in a medical procedure, retractor 50 and multitool 100 may be used separately in conjunction with a single procedure or in different medical procedures. Retractor 50 may be used to retract many different types of tissue, and, similarly, multitool instrument 100 may be used to dissect, clamp, coagulate, and cut tissues during other types of endoscopic and open surgical procedures. For example, the instruments can also be used to remove other discrete tissues, such as tumors, to ligate fallopian tubes for fertility control, to ligate and transect bile ducts for nephrectomy, or to transect ligaments or other tissue structures.

While there has been shown and described what is considered to be preferred embodiments of the invention, it will, of course, be understood that various modifications and changes in form or detail could readily be made without departing from the spirit of the invention. For example, while handle 51 is depicted as an L-shaped handle, the handle could be an in-line handle, which is well-known in the art. And, while multitool 100 is shown having a single button 115, alternatively two buttons can be provided. One button can be provided to move tube 304 between the proximal and distal positions and a second button can move anvil 308 between the open and closed positions and move cutting blade 314 between the proximal and distal positions. Furthermore, a switch (not shown) can be provided to apply the cauterization energy to the electrodes automatically upon the completion of clamping of the tissue and subsequent to the cutting of the cauterized tissue. It is therefore intended that the invention be not limited to the exact forms described and illustrated, but should be constructed to cover all modifications that may fall within the scope of the appended claims.

1. A surgical end effector, comprising:
an anvil movable between a first position and a second position, the anvil having a concave anvil surface; and
a convex mating surface; wherein the anvil clamps a tissue structure between the anvil surface and the mating surface when in the second position, and wherein the mating surface has a different radius of curvature than the anvil surface.

2. The device of claim 1, wherein the mating surface radius curvature is greater than the anvil surface radius curvature.

3. The device of claim 2, wherein the anvil surface has a curvature radius of approximately 0.11 inches and the mating surface has a curvature radius of approximately 0.12 inches.

4. The device of claim 2, wherein the mating surface has a curvature radius ranging from 5% to 20% larger than the curvature radius of the anvil surface.

5. The device of claim 4, wherein the mating surface has a curvature radius approximately 10% larger than the curvature radius of the anvil surface.

6. The device of claim 1, comprising a shaft having a lumen and an opening in a distal end, and wherein the mating surface is located distal of the opening.

7. The device of claim 6, wherein the mouth of the opening is approximately 5 mm.

8. The device of claim 6, wherein the mating surface is a portion of a tip disposed at least partially within the shaft lumen, and the shaft is configured such that the shaft surface contacts the mating surface at a location distal of the opening.

9. The device of claim 8, wherein the shaft includes a distal portion that is oblong in shape.

10. The device of claim 1, comprising:
a shaft having a lumen and an opening in a distal end;
a tip disposed at least partially within the shaft lumen, the tip having a proximal portion and a distal portion, and wherein the mating surface forms a surface of the proximal portion.

11. The device of claim 10, wherein the distal portion extends beyond the distal end of the shaft.

12. The device of claim 10, wherein the distal portion includes two outer portions and a narrower, middle portion disposed within the two outer portions.

13. The device of claim 1, wherein the anvil applies a clamping pressure between 280 psi and 420 psi.
